SEQUENCE LISTING (4 sequences)

SEQ ID NO: 1 - DNA, 19 bp, Artificial Sequence
  primer
gaaggtgaaggtcggagtc

SEQ ID NO: 2 - DNA, 20 bp, Artificial Sequence
  primer
gaagatggtgatgggatttc

SEQ ID NO: 3 - DNA, 20 bp, Artificial Sequence
  primer
tcattcgagtagcggctctt

SEQ ID NO: 4 - DNA, 20 bp, Artificial Sequence
  primer
cttctttgctcctccattgc

(19) United States
(12) Patent Application Publication
(10) Pub. No.: US 2011/0244461 A1
(43) Pub. Date: Oct. 6, 2011

(54) METHOD FOR PREPARING STOOL SAMPLE, SOLUTION FOR PREPARING STOOL SAMPLE AND STOOL COLLECTION KIT

(75) Inventors: Yasuo Tanigami, Tokyo (JP); Tomonori Nagaoka, Tokyo (JP)

(73) Assignee: OLYMPUS CORPORATION, Tokyo (JP)

(21) Appl. No.: 13/152,908

(22) Filed: Jun. 3, 2011

Related U.S. Application Data
(63) Continuation of application No. PCT/JP2009/070186, filed on Dec. 1, 2009.

(30) Foreign Application Priority Data
Dec. 5, 2008 (JP) 2008-310988

(51) Int. Cl. C12Q 1/68; C07H 1/08

(52) U.S. Cl. 435/6.11; 435/6.1; 536/25.4; 536/25.42; 435/6.14; 435/6.18

(57) ABSTRACT

The present invention relates to the providing of a method for preparing a stool sample that enables a nucleic acid in a stool to be stably preserved without requiring a complex procedure, a solution for preparing a stool sample, a stool collection kit used in that method, and a method for recovering and analyzing a nucleic acid in a stool using a stool sample prepared using the preparation method of the present invention. A method for preparing a stool sample according to the present invention is a method for preparing a stool sample being used for analyzing a nucleic acid contained in the stool, and is characterized in that a collected stool is mixed with a solution having a protease inhibitor as an active ingredient.

The present continuation application is on PCT International Patent Application No. PCT/JP2009/070186, which claims priority on the basis of Japanese Patent Application No. 2008-310988, filed in Japan on Dec. 5, 2008, the contents of which are incorporated herein by reference.

### TECHNICAL FIELD

The present invention relates to a method for preparing a stool sample in order to analyze a nucleic acid contained in the stool sample, a solution for preparation a stool sample and a kit for collecting stool, a stool sample prepared by the above preparation method, a method for recovering a nucleic acid from the above stool sample, and a method for analyzing a nucleic acid that uses a nucleic acid recovered according to the above nucleic acid recovering method.

### BACKGROUND ART

The number of colorectal cancer patients is currently continuing to increase rapidly each year in not only the U.S. and Europe, but in Japan as well, and is becoming one of the leading causes of cancer-related deaths. This is thought to be due to the growing proliferation of a Western style diet consisting primarily of red meat among the Japanese people. More specifically, roughly 60,000 persons are afflicted with colorectal cancer each year, and in terms of the number of deaths by organ, colorectal cancer is ranked third after gastric cancer and lung cancer, and is predicted to continue to increase in the future. On the other hand, differing from other forms of cancer, colorectal cancer has a nearly 100% cure rate if treated soon after onset. Thus, it is extremely significant to include colorectal cancer in early cancer screening examinations, and research and development of testing methods for early discovery of colorectal cancer is proceeding at a rapid pace.

Methods such as barium enema examinations and colonoscopies are performed as testing methods for early discovery of colorectal cancer. Barium enema examinations consist of injecting barium into the large intestine and allowing it to adhere to the mucosal membranes of the large intestine, irradiating the intestine with X-rays to capture images of any surface irregularities, and then observing the surface.

On the other hand, colonoscopy consists of observing the inside of the large intestine directly with an endoscope. Colonoscopy in particular enables high levels of sensitivity and specificity, while also offering the advantage of allowing the excision of polyps and early forms of cancer.

However, in addition to be associated with high costs, these examinations place a considerable burden on the patient while also having the problem of being accompanied by complication risks. For example, barium enemas have risks associated with X-ray exposure and intestinal obstruction. In addition, colonoscopy is an invasive procedure since the endoscope is inserted directly into the large intestine. Moreover, the endoscopic procedure requires an experienced technician and the number of facilities where this examination can be performed is limited. Consequently, these examinations are not suitable for colorectal cancer examinations targeted at asymptomatic, healthy individuals as part of routine health examinations and the like.

In recent years, fecal occult blood tests have been widely performed as a non-invasive and inexpensive method for primary screening for colorectal cancer. The fecal occult blood test is a test for the presence of hemoglobin originating in erythrocytes contained in fecal matter, and is used as a method for indirectly predicting the presence of colorectal cancer. Factors behind the widespread use of the fecal occult blood test include stool samples being able to be collected and stored at room temperature eliminating the need for refrigerators, freezers and other special storage conditions, samples being able to be collected easily at home, and the test procedure being extremely simple. However, since the fecal occult blood test has low sensitivity of only about 25%, it has the problem of a high percentage of colorectal cancer being overlooked. Moreover, it also has a low positive predictive value, with the percentage of actual colorectal cancer patients among subjects judged to be positive in the fecal occult blood test being only about 10%, thus resulting in a large number of false positives. Consequently, there is a strong need for the development of a new examination method offering higher reliability.

Attention is currently focusing on new examination methods that are suitable for routine health examinations by being non-invasive, simple and highly reliable for use in testing for the presence of cancer cells and cancer cell-derived genes in stool samples. Since these examination methods investigate the presence of cancer cells or cancer cell-derived genes directly, they are considered to be more reliable than the fecal occult blood test, which tests for the presence of blood from the digestive tract that occurs indirectly accompanying the onset of colorectal cancer.

In order to accurately detect cancer cells and the like in stool samples, it is important to efficiently recover cancer cell-derived nucleic acids from those stool samples. In particular, since cancer cell-derived nucleic acids are only present in trace amounts in stool samples, and stool samples also contain large amounts of digestive remnants and bacteria, nucleic acids are decomposed extremely easily. Consequently, in order to efficiently recover nucleic acids, and particularly nucleic acids derived from mammalian cells such as human cells, from stool samples, it is important to prevent decomposition of nucleic acids within the stool and prepare the stool sample so that it can be stored stably until the time of the testing procedure. An example of such a stool sample processing method consists of separating cancer cells that have exfoliated from the large intestine or other constituent of the digestive tract from a collected stool sample. Separation of cancer cells from stool makes it possible to inhibit the effects of bacterial proteases, DNase, RNase and other degrading enzymes. Examples of methods that have been disclosed for separating cancer cells from stool include: (1) a method for separating cells from stool, comprising: (a) a step for cooling the stool to a temperature below its gel freezing point, and (b) a step for collecting cells from the stool while maintaining at a temperature below the gel freezing point so that the stool substantially remains completely intact (see, for example, Japanese Translation of PCT Application No. H11-511982). Another example of such a method consists of: (2) dispersing the stool in a transport medium containing a protease inhibitor, mucous dissolver and bactericide at a normal ambient temperature, followed by isolating the colorectal exfoliated cells (see, for example, Japanese Translation of PCT Application No. 2004-519202).

On the other hand, numerous fixation methods, such as formalin fixation or alcohol fixation, have conventionally been employed to maintain the morphology of collected cells until the time of observation in cases of histological and cytological observation of cell morphology. A method that has been disclosed as an example of a method that applies these fixation methods consists of (3) a cell solution preservative comprising an alcohol that is miscible with an amount of water sufficient for colonizing mammalian cells, an amount of anti-aggregation agent sufficient for preventing aggregation of mammalian cells in the solution, and a buffer for maintaining the pH of the solution within a range of 4 to 7 during the time the cells are stored, which is used as a storage solution for enabling mammalian cell samples to be stored for long periods of time or enable cells to be observed following storage (see, for example, Patent Japanese Unexamined Patent Application, First Publication No. 2003-153688).

In addition, disclosed examples of storage solutions that enable histological and cytological examinations of cells as well as molecular analyses of proteins or nucleic acids and the like present in cells after storage include (4) a universal collection medium containing a buffer component, at least one alcohol component, a fixative component and a chemical agent that inhibits decomposition of at least one member selected from the group consisting of RNA, DNA and protein (see, for example, Japanese Translation of PCT Application No. 2004-500897), and a non-aqueous solution containing 5 to 20% polyethylene glycol and 80 to 95% methanol (see, for example, Japanese Translation of PCT Application No. 2005-532824). In addition, (6) a composition has been disclosed for stabilizing cell structure and nucleic acids that comprises (a) a first substance capable of precipitating or denaturing protein containing at least one member of alcohol or ketone, and (b) a second promoting substance for promoting injection of the first substance into at least one cell (see, for example, Japanese Unexamined Patent Application, First Publication No. 2001-128662).

Furthermore, examples of methods that have been disclosed for stabilizing cell-derived components in stool include: (7) a collection container including at least a single type of stabilizing agents such as protease inhibitors of which amount is sufficient for inhibiting decomposition and/or fragmentation of proteins, and a method for collecting biological samples, especially whole blood, with using the collection container (see, for example, Japanese Translation of PCT Application No. 2005-525126) , (8) a method for stabilizing cells and nucleic acids in a sample by exposing the sample to a composition which has inhibitory effect on protein-inhibitory compounds and/or nucleic acid-inhibitory compounds in the sample (see, for example, Japanese Unexamined Patent Application, First Publication No. 2004-159648), (9) a method for preserving the integrity of DNA in a stool sample comprising a step of exposing the stool sample to a sufficient amount of inhibitors of DNA-degradation (see, for example, Japanese Translation of PCT Application No. 2002-537777).

### SUMMARY OF THE INVENTION

As a result of intensive and extensive studies in order to solve the above-mentioned problems, the inventors of the present invention found that a stool sample enabling stable storage of a nucleic acid contained in stool was able to be prepared by mixing a collected stool with a solution for preparing a stool sample having a protease inhibitor as an active ingredient thereof, especially with a water-soluble organic solvent containing a protease inhibitor, thereby leading to completion of the present invention.

Namely, the present invention includes the following aspects.

(1) A method for preparing a stool sample, comprising: mixing a collected stool with a solution for preparing a stool sample having a protease inhibitor as an active ingredient, wherein the stool sample is used for analyzing a nucleic acid contained in the stool.

(2) In the method for preparing a stool sample according to the aspect (1), it is preferred that the mixture of the stool and the solution for preparing a stool sample be stored for a predetermined amount of time.

(3) In the method for preparing a stool sample according to the aspect (2), it is preferred that the amount of time during which the mixture is stored be 1 hour or more.

(4) In the method for preparing a stool sample according to any one of the aspects (1) to (3), it is preferred that the protease inhibitor be one or more members selected from the group consisting of a peptidic protease inhibitor, a reducing agent, a protein denaturing agent, and a chelating agent.

(5) In the method for preparing a stool sample according to any one of the aspects (1) to (3), it is preferred that the protease inhibitor be one or more members selected from the group consisting of AEBSF, Aprotinin, Bestain, E-64, Leupeptin, Pepstatin, DTT(dithiothreitol), and EDTA.

(6) In the method for preparing a stool sample according to any one of the aspects (1) to (5), it is preferred that the solution for preparing a stool sample further contain a water-soluble organic solvent as an active ingredient.

(7) In the method for preparing a stool sample according to any one of the aspects (1) to (6), it is preferred that the solution for preparing a stool sample have a buffering action.

(8) In the method for preparing a stool sample according to any one of the aspects (1) to (7), it is preferred that the pH of the solution for preparing a stool sample be from 2 to 6.5.

(9) In the method for preparing a stool sample according to any one of the aspects (6) to (8), it is preferred that the water-soluble organic solvent be one or more members selected from the group consisting of a water-soluble alcohol, ketone and aldehyde.

(10) In the method for preparing a stool sample according to any one of the aspects (6) to (8), it is preferred that the water-soluble organic solvent be one or more members selected from the group consisting of a water-soluble alcohol and ketone, and that the concentration of the water-soluble organic solvent is 30% or more.

(11) In the method for preparing a stool sample according to any one of the aspects (6) to (10), it is preferred that the water-soluble organic solvent contain one or more members selected from the group consisting of ethanol, propanol and methanol as water-soluble alcohol.

(12) In the method for preparing a stool sample according to any one of the aspects (6) to (11), it is preferred that the water-soluble organic solvent be ethanol.

(13) In the method for preparing a stool sample according to any one of the aspects (6) to (11), it is preferred that the water-soluble organic solvent contain one or more members selected from the group consisting of acetone and methyl ethyl ketone as ketone.

(14) In the method for preparing a stool sample according to any one of the aspects (6) to (8), it is preferred that the water-soluble organic solvent be an aldehyde, and the concentration of the water-soluble organic solvent is within a range of 0.01 to 30%.

(15) In the method for preparing a stool sample according to any one of the aspects (1) to (14), it is preferred that in terms of a mixing ratio of the stool and the solution for preparing a stool sample, a volume of the solution for preparing the stool sample be one or more relative to 1 volume of the stool.

(16) In the method for preparing a stool sample according to any one of the aspects (2) to (15), it is preferred that the amount of time during which the mixture be stored is 12 hours or more.

(17) In the method for preparing a stool sample according to any one of the aspects (2) to (15), it is preferred that the amount of time during which the mixture be stored is 24 hours or more.

(18) In the method for preparing a stool sample according to any one of the aspects (2) to (15), it is preferred that the amount of time during which the mixture be stored is 72 hours or more.

(19) In the method for preparing a stool sample according to any one of the aspects (8) to (18), it is preferred that the pH of the solution for preparing a stool sample be from 3 to 6.

(20) In the method for preparing a stool sample according to any one of the aspects (8) to (18), it is preferred that the pH of the solution for preparing a stool sample be from 4.5 to 5.5.

(21) In the method for preparing a stool sample according to any one of the aspects (1) to (20), it is preferred that the solution for preparing a stool sample further contain a surface active agent.

(22) In the method for preparing a stool sample according to any one of the aspects (1) to (21), it is preferred that the solution for preparing a stool sample further contain a colorant.

(23) A solution for preparing a stool sample that is used to mix a collected stool, comprising: a protease inhibitor as an active ingredient, wherein the stool sample being used for recovering a nucleic acid from the stool sample.

(24) In the solution for preparing a stool sample that is used to mix a collected stool according to the aspect (23), it is preferred that the solution for preparing a stool sample further contain a water-soluble organic solvent as an active ingredient.

(25) In the solution for preparing a stool sample that is used to mix a collected stool according to the aspect (23) or (24), it is preferred that the protease inhibitor be one or more members selected from the group consisting of a peptidic protease inhibitor, a reducing agent, a protein denaturing agent, and a chelating agent.

(26) In the solution for preparing a stool sample that is used to mix a collected stool according to the aspect (24) or (25), it is preferred that the water-soluble organic solvent be one or more members selected from the group consisting of a water-soluble alcohol and ketone.

(27) A stool collection kit, comprising: a stool collection container; and a solution for preparing a stool sample having a protease inhibitor as an active ingredient, wherein the stool collection container includes the solution for preparing a stool sample.

(28) A stool sample prepared by the method for preparing a stool sample according to any one of the aspects (1) to (22).

(29) A method for recovering a nucleic acid from a stool sample comprising: simultaneously recovering a nucleic acid derived from indigenous intestinal bacterium and a nucleic acid derived from an organism other than indigenous intestinal bacterium, from the stool sample, and the stool sample is prepared by mixing a collected stool with a solution for preparing a stool sample having a protease inhibitor as an active ingredient.

(30) In the method for recovering a nucleic acid from a stool sample according to the aspect (29), it is preferred that the nucleic acid derived from the organism other than indigenous intestinal bacterium be the nucleic acid derived from a mammalian cell.

(31) In the method for recovering a nucleic acid from a stool sample according to the aspect (29) or (30), it is preferred that the step for recovering a nucleic acid include:

(a) a step for denaturing a protein in the stool sample and thereby extracting a nucleic acid from indigenous intestinal bacterium and an organism other than indigenous intestinal bacterium in the stool sample; and

(b) a step for recovering the nucleic acid extracted in the step (a).

(32) In the method for recovering a nucleic acid from a stool sample according to the aspect (31), it is preferred that the step for recovering a nucleic acid include further, following the step (a) and prior to the step (b),

(c) a step for removing the protein denatured in the step (a).

(33) In the method for recovering a nucleic acid from a stool sample according to the aspect (31) or (32), it is preferred that denaturing of a protein in the step (a) be carried out using one or more materials selected from the group consisting of a chaotropic salt, an organic solvent and a surface active agent.

(34) In the method for recovering a nucleic acid from a stool sample according to the aspect (33), it is preferred that the organic solvent be phenol.

(35) In the method for recovering a nucleic acid from a stool sample according to any one of the aspects (32) to (34), it is preferred that the removal of denatured protein in the step (c) be carried out using chloroform.

(36) In the method for recovering a nucleic acid from a stool sample according to any one of the aspects (31) to (35), it is preferred that the recovery of nucleic acid in the step (b) include:

(b1) a step for adsorbing the nucleic acid extracted in the step (a) to an inorganic support, and

(b2) a step for eluting the nucleic acid adsorbed in the step (b1) from the inorganic support.

(37) In the method for recovering a nucleic acid from a stool sample according to any one of the aspects (31) to (36), it is preferred that the step for recovering a nucleic acid include further, prior to the step (b),

(d) a step for recovering a solid component from the stool sample.

(38) A method for analyzing a nucleic acid comprising:

conducting an analysis of a nucleic acid derived from a mammalian cell,

wherein the nucleic acid is recovered from a stool sample by use of the method for recovering a nucleic acid according to any one of the aspects (29) to (37).

(39) In the method for analyzing a nucleic acid according to the aspect (38), it is preferred that the mammalian cell be a gastrointestinal tract cell.

(40) In the method for analyzing a nucleic acid according to the aspect (38), it is preferred that the mammalian cell be a cell exfoliated from a large intestine.

(41) In the method for analyzing a nucleic acid according to any one of the aspects (38) to (40), it is preferred that the nucleic acid derived from a mammalian cell be a marker indicating a neoplastic transformation.

(42) In the method for analyzing a nucleic acid according to any one of the aspects (38) to (40), it is preferred that the nucleic acid derived from a mammalian cell be a marker indicating an inflammatory gastrointestinal disease.

(43) In the method for analyzing a nucleic acid according to any one of the aspects (38) to (40), it is preferred that the nucleic acid derived from a mammalian cell be a nucleic acid derived from COX-2 gene.

(44) In the method for analyzing a nucleic acid according to any one of the aspects (38) to (43), it is preferred that the analysis be one or more of RNA analysis and DNA analysis.

(45) In the method for analyzing a nucleic acid according to the aspect (44), it is preferred that the RNA analysis be one or more analysis selected from the group consisting of an analysis for insertion, deletion, substitution, duplication or inversion of one or more bases in the RNA, an analysis for a splicing variant, an mRNA expression analysis, and a functional RNA analysis.

(46) In the method for analyzing a nucleic acid according to the aspect (44), it is preferred that the DNA analysis be one or more of a mutation analysis and an analysis of an epigenetic change.

(47) In the method for analyzing a nucleic acid according to the aspect (46), it is preferred that the mutation analysis be an analysis for one or more mutations of an insertion, deletion, substitution, duplication or inversion of one or more bases.

(48) In the method for analyzing a nucleic acid according to the aspect (46), it is preferred that the analysis of an epigenetic change be one or more of a DNA methylation analysis and a DNA demethylation analysis.

(49) In the method for analyzing a nucleic acid according to the aspect (46), it is preferred that the mutation analysis be a mutation analysis of a K-ras gene.

### BRIEF DESCRIPTION OF THE DRAWINGS

FIG. 1 is a diagram showing an embodiment of a stool collection container which can be used for a stool collection kit according to the present invention.

FIGS. 2(a)-(f) are diagrams showing an embodiment of a stool collection container which can be used for a stool collection kit according to the present invention.

FIG. 3 is a graph showing the results of a relative comparison of expressed amounts of GAPDH gene in RNA derived from stool samples 1-1 to 1-5 in Example 1.

FIG. 4 is a graph showing the results of a relative comparison of expressed amounts of GAPDH gene in RNA derived from stool samples 2-1 to 2-3 in Example 2.

FIG. 5 is a graph showing amounts of RNA recovered from each of the stool samples in Reference Example 1.

FIG. 6 is a graph showing amounts of RNA recovered from stool samples prepared using ethanol solutions of various concentrations in Reference Example 3.

### BEST MODE FOR CARRYING OUT THE INVENTION

A method for preparing a stool sample according to the present invention is a method for preparing a stool sample which is used for analyzing cells or cell-derived components contained in stool, especially nucleic acids, and is characterized in that the collected stool is mixed with a solution for preparing a stool sample having a protease inhibitor as an active ingredient of the solution. In the present invention, in order to improve the preservation stability of cells or cell-derived components in stool, especially nucleic acids, a protease inhibitor is used as an active ingredient instead of an inhibitor of nucleic acid decomposition. Immediately after the excretion, cell-derived components such as nucleic acids usually exist inside cells in stool, but they are released outside cells through holes and the like in plasma membrane formed as a result of decomposition of proteins and the like by proteases which exist abundantly in the stool. After that, the cell-derived components such as nucleic acids released outside cells are decomposed by nucleases and the like which also exist abundantly in the stool. In the present invention, preservation stability of cell-derived components is improved by using a protease inhibitor as an active ingredient to effectively inhibit the decomposition of plasma membrane protein and to keep them inside cells.

The solution for preparing a stool sample used in the method for preparing a stool sample according to the present invention (to also be referred to as the “solution for preparing of the present invention”) contains a protease inhibitor as an active ingredient of the solution. The protease inhibitor is not particularly limited as long as it has the ability to inhibit enzyme activity of protease (an enzyme which has the ability to hydrolyze a peptidic bond). Examples of the protease inhibitor include proteinase inhibitors and peptidase inhibitors. Examples also include agents having inhibitory activity against serine protease, agents having inhibitory activity against cysteine protease, agents having inhibitory activity against aspartic protease (acidic protease), and agents having inhibitory activity against metallo protease.

As the protease inhibitor used in the present invention, a suitably selected protease inhibitor from conventionally known protease inhibitors can be used. Examples of the protease inhibitor used in the present invention include AEBSF, Aprotinin, Bestain, Calpain Inhibitor I, Calpain Inhibitor II, Chymostatin, 3,4-Dichloroisocoumain, E-64, Lactacystin, Leupeptin, MG-115, MG-132,PepstatinA, PMSF, Proteasome Inhibitor, TLCK, TPCK, and Trypsin Inhibitor. In addition, the mixture of a number of protease inhibitors, which is generally called as “protease inhibitor cocktail” can be used.

The concentration of protease inhibitor in the solution for preparing of the present invention is not particularly limited as long as it is a concentration capable of inhibiting proteases derived from stool in the stool sample, and thus can be appropriately determined in consideration of the types of protease inhibitor, the pH value or temperature of the solution for preparing a stool sample, a mixing ratio of the stool and the solution for preparing the stool sample, or the like. Preferable concentration of each protease inhibitor in the solution for preparing the stool sample is shown in Table 1.

TABLE 1

| Protease inhibitor | concentration | |
|---|---|---|
| AEBSF | 0.1~1.0 | mg/ml |
| Aprotinin | 0.06~2 | μg/ml |
| Bestain | 4~400 | μg/ml |
| Calpain Inhibitor I | 1~100 | μg/ml |
| Calpain Inhibitor II | 1~100 | μg/ml |
| Chymostatin | 6~60 | μg/ml |
| 3,4-Dichloroisocoumain | 1~43 | μg/ml |
| E-64 | 0.5~10 | μg/ml |
| Lactacystin | 1~100 | μM |
| Leupeptin | 0.1~10 | μg/ml |
| MG-115 | 0.1~10 | μM |
| MG-132 | 0.1~10 | μM |
| PepstatinA | 0.7 | μg/ml |
| PMSF | 17~170 | μg/ml |
| Proteasome Inhibitor | 0.1~10 | μM |
| TLCK | 37~50 | μg/ml |
| TPCK | 70~100 | μg/ml |
| Trypsin Inhibitor | 10~100 | μg/ml |

The protease inhibitor used in the present invention may be peptidic protease inhibitor as mentioned above, a reducing agent, a protein denaturing agent, or a chelating agent. In the present invention, the term “peptidic protease inhibitor” refers to a peptide or a modified peptide which has an ability to interact with a protease and to inhibit protease activity of the protease.

Examples of the chelating agent used in the present invention include ethylendiaminetetraacetic acid (EDTA), O,O′-bis(2-aminophenyl)ethylene glycol-N,N,N′,N′-tetraacetic acid (BAPTA), N,N-Bis(2-hydroxyethyl) glycine (Bicine), trans-1,2-diaminocyclohexane-ethylendiaminetetraacetic acid (CyTDA), 1,3-diamino-2-hydroxypropane-tetraacetic acid (DPTA-OH), diethylene-triamine-pentaacetic acid (DTPA), ethylendiamine dipropanoic acid hydrochloride, ethylendiamine-2-methylene phosphonic acid hydrate (EDDPO), N-(2-hydroxyethyl)ethylendiamine trisacetic acid (EDTA-OH), ethylendiamine tetra(methylene phosphonic acid) (EDTPO), O,O′-bis(2-aminoethyl)ethylene glycol tetraacetic acid (EGTA), N,N-bis (2-hydroxybenzyl)ethylenediamine diacetic acid (HBED), 1,6-hexamethylenediamine tetraacetic acid (HDTA), N-(2-hydroxyethyl)iminodiacetic acid (HIDA), iminodiacetic acid (IDA), 1,2-diaminopropane tetraacetic acid (Methyl-EDTA), nitrilotriacetic acid (NTA), nitrilotripropanoic acid (NTP), nitrilotris(methylphosphonic acid), trisodium salt (NTPO), N,N,N′,N′-tetrakis(2-pyridylmethyl)ethylenediamine (TPEN), and triethylene tetraamine-hexaacetic acid (TTHA).

The concentration of a chelating agent as a protease inhibitor in the solution for preparing of the present invention is not particularly limited as long as it is a concentration capable of inhibiting proteases derived from stool in the stool sample, and thus can be appropriately determined in consideration of the types of chelating agents, or the like. It is preferred that each chelating agent is added so that the final concentration in the solution for preparing the stool sample is within a range of 0.1 mM to 1 M.

Examples of the reducing agent used in the present invention include DTT (dithiothreitol), and β mercaptoethanol.

The concentration of reducing agents as a protease inhibitor in the solution for preparing of the present invention is not particularly limited as long as it is a concentration capable of inhibiting proteases derived from stool in the stool sample, and thus can be appropriately determined in consideration of the types of reducing agents, or the like. It is preferred that each reducing agent is added so that the final concentration in the solution for preparing a stool sample is within a range of 0.1 mM to 1 M.

Examples of the protein denaturing agent used in the present invention include urea, guanine, and guanidinium hydrochloride.

The concentration of a protein denaturing agent as a protease inhibitor in the solution for preparing of the present invention is not particularly limited as long as it is a concentration capable of inhibiting proteases derived from stool in the stool sample, and thus can be appropriately determined in consideration of the types of protein denaturing agent, or the like. It is preferred that each protein denaturing agent is added so that the final concentration in the solution for preparing a stool sample is within a range of 0.1 mM to 1 M.

The solution for preparing of the present invention may contain only a single type of protease inhibitor or may contain two or more types of protease inhibitors. For example, it may contain two or more types of peptidic protease inhibitors such as AEBSF in combination, and may contain different types of protease inhibitors such as a combination of a peptidic protease inhibitor and a chelating agent, and as a combination of a peptidic protease inhibitor and a reducing agent.

In addition to a protease inhibitor, the solution for preparing of the present invention preferably contains a water-soluble organic solvent as an active ingredient of the solution. By mixing a stool into the water-soluble organic solvent containing a protease inhibitor, loss of nucleic acids contained in the stool due to decomposition and the like can be held to a minimum, and nucleic acids can be stored extremely stably in the water-soluble organic solvent. The reason why the water-soluble organic solvent containing a protease inhibitor demonstrates this improved effect of nucleic acid preservation is speculated that the dehydrating action possessed by the water-soluble organic solvent would lower considerably cellular activity of organisms having nucleic acids such as indigenous intestinal bacteria, mammalian cells or viruses, thereby changes in nucleic acids. The reason is also speculated that the protein denaturing action possessed by the water-soluble organic solvent component would decrease considerably the activities of various decomposing enzymes such as protease, DNase or RNase present in stool, thereby decomposition of cell-derived components such as nucleic acid inhibited.

Biological samples such as stool usually contain a large amount of water. Therefore, due to using a water-soluble organic solvent, which is a solvent having high water solubility or a solvent capable of mixing at an arbitrary ratio with water, as an active ingredient, the solution for preparing of the present invention is able to rapidly mix with a stool, thereby further increasing the efficiency of nucleic acid recovery.

In the present invention, the term “water-soluble organic solvent” refers to alcohols, ketones, aldehydes, and combinations of these solvents, and these solvents have straight chain structures and are in a liquid state at a temperature close to room temperature, for example, from 15° C. to 40° C. By containing a water-soluble organic solvent with a straight chain structure as an active ingredient, the solution is able to mix with stool more rapidly than by containing a water-soluble organic solvent with a cyclic structure such as a benzene ring as an active ingredient. Since organic solvents having a cyclic structure typically easily separate from water, they do not easily mix with stool and it is difficult for them to obtain an improved effect of nucleic acid preservation. This is because, even in the case of a solvent which is soluble in water to a certain extent, in order to homogeneously disperse stool therein, stool samples need to be mixed vigorously or be heated in many cases. In order to make the mixing of the organic solvents having a cyclic structure with stool easier, it is also possible to prepare a mixed solution of organic solvents and water in advance, followed by the mixing of stool with the mixed solution. However, for preparing the mixed solution, the organic solvents having a cyclic structure and water need to be mixed vigorously or be heated in many cases, which is not preferable.

In the solution for preparing of the present invention, the water-soluble organic solvent preferably has a water solubility of 12% by weight or more, more preferably a water solubility of 20% by weight or more, still more preferably a water solubility of 90% by weight or more, and it is most preferable that the water-soluble organic solvent be one which can be mixed with water at a given ratio. Examples of the water-soluble organic solvent which can be mixed with water at a given ratio include methanol, ethanol, n-propanol, 2-propanol, acetone and formaldehyde.

The water-soluble organic solvent contained in the solution for preparing of the present invention is not particularly limited as long as it satisfies the above definition and is a solvent that demonstrates an improved effect of nucleic acid preservation. Examples of the water-soluble organic solvent include alcohols which are water-soluble alcohols such as methanol, ethanol, propanol, butanol and mercaptoethanol; ketones such as acetone and methyl ethyl ketone (having a water solubility of 90% by weight); aldehydes such as acetaldehyde (acetyl aldehyde), formaldehyde (formalin), glutaraldehyde, paraformaldehyde and glyoxal. Propanol may be either n-propanol or 2-propanol. Further, butanol may be either 1-butanol (having a water solubility of 20% by weight) or 2-butanol (having a water solubility of 12.5% by weight). The water-soluble organic solvent used in the present invention is preferably a water-soluble alcohol, acetone, methyl ethyl ketone or formaldehyde. This is because these solvents have sufficiently high water solubility. From the viewpoints of availability, handling ease, safety and the like, the water-soluble organic solvent is more preferably a water-soluble alcohol, and still more preferably ethanol, propanol or methanol. In particular, ethanol is particularly useful in the screening test for routine health examinations or the like since it is the safest and can easily be handled even in the home.

The concentration of the water-soluble organic solvent in the solution for preparing of the present invention is not particularly limited as long as it is a concentration that demonstrates an improved effect of nucleic acid preservation, and thus can be appropriately determined in consideration of the types of water-soluble organic solvent, or the like. For example, when a water-soluble alcohol or ketone is used as an active ingredient, the concentration of the water-soluble organic solvent in the solution for preparing of the present invention is preferably 30% or more. If the concentration of the water-soluble organic solvent is sufficiently high, when a stool and the solution for preparing a stool sample are mixed, the water-soluble organic solvent component rapidly penetrates into mammalian cells and indigenous intestinal bacteria in the stool, thereby enabling an improved effect of nucleic acid preservation to be demonstrated rapidly.

Note that in the present invention and in the present description of the present application, the term “%” refers to “% by volume (vol %)”, unless otherwise specified.

In particular, when a water-soluble alcohol is used as an active ingredient, the concentration of the water-soluble organic solvent in the solution for preparing of the present invention is preferably 30% or more, more preferably 50% or more, still more preferably within a range of 50 to 80%, and most preferably within a range of 60 to 70%. If the concentration of the water-soluble organic solvent is high, even when a stool contains a large amount of water, sufficiently high effect of nucleic acid preservation can be achieved by adding a small amount of the solution for preparing a stool sample to the stool.

When acetone or methyl ethyl ketone is used as an active ingredient, the concentration of the water-soluble organic solvent in the solution for preparing of the present invention is preferably 30% or more, more preferably 60% or more, and still more preferably 80% or more. Alternatively, when acetaldehyde, formaldehyde, glutaraldehyde, paraformaldehyde or glyoxal is used as an active ingredient, the concentration of the water-soluble organic solvent in the solution for preparing of the present invention is preferably within a range of 0.01 to 30%, more preferably within a range of 0.03 to 10%, and still more preferably within a range of 3 to 5%. Aldehydes are able to demonstrate an improved effect of nucleic acid preservation at lower concentrations than alcohols or ketones.

In addition, the water-soluble organic solvent used in the present invention may only contain a single type of water-soluble organic solvent or may be a mixed solution of two or more types of water-soluble organic solvents. For example, the water-soluble organic solvent may be a mixed solution of two or more types of alcohols, or may be a mixed solution of an alcohol and another type of water-soluble organic solvent. A mixed solution of alcohol and acetone is preferable since nucleic acid storage efficiency is further improved.

The pH of the solution for preparing of the present invention is preferably acidic. This is to more effectively inhibit hydrolysis of nucleic acids. The pH of the solution for preparing of the present invention is preferably within a range of 2 to 6.5, more preferably within a range of 3 to 6, and still more preferably within a range of 4.5 to 5.5.

The solution for preparing of the present invention preferably has buffering action so that the pH thereof fluctuates a little and is maintained within the aforementioned pH ranges after adding a certain amount of acid or base, particularly a stool, to the solution. As the solution for preparing a stool sample having buffering action, a solution prepared by adding the active ingredient such as a protease inhibitor and a water-soluble organic solvent to an appropriate buffer solution may be used. In this invention, the solution for preparing a stool sample preferably contains an organic acid and a conjugate base of that organic acid and demonstrates buffering action attributable to the organic acid and the conjugate base thereof. For example, the solution for preparing a stool sample may be adjusted to a desired pH by adding an organic acid and an alkaline metal salt or alkaline earth metal salt of that organic acid. The pH thereof may also be adjusted by using a hydroxide of an alkaline metal or alkaline earth metal after adding the organic acid.

In addition, the solution for preparing of the present invention may be a solution that contains both organic acid and mineral acid and has suitable buffering action. For example, the solution for preparing a stool sample may be a solution obtained by mixing a water-soluble organic solvent with a buffer system having buffering action in the acidic range such as a glycine/HCl buffer system, sodium cacodylate/HCl buffer system or potassium hydrogen phthalate/HCl buffer system.

In the present invention, the pH of the solution for preparing a stool sample is the value obtained by measuring with a pH meter of which the measuring principle is the glass electrode method (such as that manufactured by DKK-Toa Corp.) after having calibrated with a phthalate standard solution and neutral phosphate standard solution.

In addition, the solution for preparing of the present invention may contain any components other than the protease inhibitor or water-soluble organic solvent provided they do not impair an improved effect of nucleic acid preservation achieved due to the protease inhibitor and water-soluble organic solvent component. For example, the solution for preparing a stool sample may contain a chaotropic salt or a surface active agent. The containing of a chaotropic salt or a surface active agent makes it possible to more effectively inhibit cellular activity and enzyme activity of various decomposing enzymes present in stool. Examples of chaotropic salts that can be added to the solution for preparing a stool sample include guanidine hydrochloride, guanidine isothiocyanate, sodium iodide, sodium perchlorate and sodium trichloroacetate. A nonionic surface active agent is preferable for the surface active agent able to be added to the solution for preparing a stool sample. Examples of these nonionic surface active agents include Tween 80, CHAPS (3-[3-cholamidopropyl-dimethylammonio]-1-propane sulfonate), Triton X-100 and Tween 20. The type and concentration of chaotropic salt or surface active agent are not particularly limited as long as it is a component with a concentration that allows the obtaining of an improved effect of nucleic acid preservation due to the protease inhibitor in the solution for preparing a stool sample, and can be appropriately determined in consideration of the amount of stool, the methods for recovering and analyzing a nucleic acid employed afterwards, or the like.

In addition, a colorant may be added to the solution for preparing a stool sample, where appropriate. By coloring the solution for preparing a stool sample, various effects can be achieved, such as the prevention of accidental swallowing and the lightening of stool color. The colorant is preferably a coloring agent used as a food additive, and is preferably blue, green, or the like. Examples of colorants include Fast Green FCF (Green No. 3), Brilliant Blue FCF (Blue No. 1) and indigo carmine (Blue No. 2). Further, a plurality of colorants may be added as a mixture, or a single colorant may be added.

In the method for preparing a stool sample according to the present invention, mixing a collected stool with the solution for preparing of the present invention may be conducted by immersing the stool in the solution for preparing a stool sample without a particular stirring treatment. The solution for preparing of the present invention is very easy to mix with stool having a large amount of water, so if the amount and condition of the stool are suitable, the solution for preparing a stool sample penetrates sufficiently into the stool and obtains the improved effect of nucleic acid preservation even when the stool is just immersed in the solution for preparing a stool sample without particularly stirring.

To mix a collected stool with the solution for preparing of the present invention may be conducted by putting and immersing the stool in the solution for preparing a stool sample and followed by stirring. Stirring make it possible to more sufficiently disperse and suspend the stool in the solution for preparing a stool sample. In the case of putting the stool into the solution for preparing a stool sample and stirring to mix, it is preferred that the mixing is carried out conducted promptly. By dispersing the stool in the solution for preparing a stool sample promptly, the water-soluble organic solvent is able to rapidly penetrate into cells present in the stool, and the improved effect of nucleic acid preservation can be obtained quickly.

Furthermore, the method used to mix the stool and the solution for preparing a stool sample is not particularly limited as long as it is a method involving physical operations. For example, the mixing may be carried out by putting the collected stool in a sealable container in which the solution for preparing a stool sample has been contained in advance, followed by vertically inverting the container or shaking the container using a shaker, such as a vortex mixer. In addition, stool and the solution for preparing a stool sample may be mixed under the presence of particles for mixing.

A method that uses a shaker or a method that uses particles for mixing is preferable for this mixing method since the mixing can be carried out rapidly. In particular, by using a stool collection container in which particles for mixing are contained in advance, the mixing can be rapidly conducted even in environments with no special equipment such as the home.

The particles for mixing are not particularly limited as long as they are formed of compositions that do not impair an improved effect of nucleic acid preservation achieved due to the protease inhibitor component and the water-soluble organic solvent component, and are particles having hardness and specific gravity sufficient to rapidly disperse stool in the solution for preparing a stool sample by colliding with the stool. The particles may be composed of one type of material or may be composed of two or more types of materials. Examples of such particles for mixing include particles composed of glass, ceramics, plastics, latex, metals, or the like. In addition, the particles for mixing may be magnetic particles or nonmagnetic particles.

The volume of the solution for preparing a stool sample to be mixed with the collected stool is not particularly limited, in terms of the mixing ratio of the stool and the solution for preparing a stool sample, the volume of the solution for preparing a stool sample is preferably one or more, relative to 1 volume of the stool. If the volume of the solution for preparing a stool sample contained in a stool collection container is equivalent to or more than the volume of the stool, when stool is collected in the stool collection container, the stool can be completely immersed in the solution, and thus the effects of the present invention can be achieved more effectively. For example, in the case when the volume of stool and that of the solution for preparing a stool sample are equivalent, it becomes possible to reduce the weight and size of the stool collection container that contains the solution for preparing a stool sample. On the other hand, by mixing a stool with the solution for preparing a stool sample whose volume is five times or more than that of the stool, the stool can be effectively and rapidly dispersed in the solution, and the adverse effects caused by the decline of water-soluble alcohol concentration due to the water contained in the stool can also be suppressed. Since a proper balance can be achieved between the two effects; i.e., the weight reduction of a stool collection container that contains the solution for preparing a stool sample, and the improvement of stool dispersibility, the mixing ratio of the stool and the solution for preparing a stool sample is preferably within a range of 1:1 to 1:20, more preferably within a range of 1:3 to 1:10 and still more preferably about 1:5.

It should be noted that the stool supplied for the method for preparing a stool sample of the present invention is not particularly limited as long as it originates from an animal (a subject), but is preferably one that originates from a mammal, and is more preferably one that originates from a human being. For example, the stool supplied is preferably a stool of a human being collected for routine health examinations, a diagnosis or the like, but it may also be a stool from livestock wild animal, or the like. In addition, the stool may be one which has been stored for a certain period of time following the collection thereof, but is preferably one which has just been collected. Furthermore, the stool is preferably collected immediately after the excretion thereof, but may be collected after a certain period of time following the excretion thereof.

The amount of the stool supplied for the method for preparing a stool sample of the present invention is not particularly limited, but is preferably within a range of 10 mg to 1 g. If the amount of stool is too large, the collection procedure requires more effort and the size of a stool collection container also becomes too large, thereby resulting in deterioration of the handling property or the like. On the other hand, in the case when the amount of stool is too small, the number of mammalian cells, such as the cells exfoliated from the large intestine, contained in the stool is too small, and the necessary amount of nucleic acid cannot be recovered, thereby resulting in reducing the level of analytical accuracy for the target nucleic acid. In addition, since stool is heterogeneous, in other words, various kinds of components are non-uniformly present therein, the stool sample is preferably collected from various parts of the stool in order to avoid the adverse effects caused by the localization of mammalian cells.

In the method for preparing a stool sample according to the present invention, better improved effect of nucleic acid preservation can be obtained by storing the mixture of the collected stool and the solution for preparing a stool sample for a predetermined amount of time. It is speculated that it needs a certain amount of time to make the protease inhibitor penetrate into the stool sufficiently because the stool contains various components such as lipid. The duration of storage of the stool sample is not particularly limited provided it is an amount of time that allows the obtaining of an improved effect of nucleic acid preservation due to the protease inhibitor in the solution for preparing a stool sample, and thus can be appropriately determined in consideration of the type and concentration of the protease inhibitor, the type and concentration of the water-soluble organic solvent, the mixing ratio of the stool and the solution for preparing a stool sample, the storage temperature, or the like. In the method for preparing a stool sample according to the present invention, the storage time of the stool sample is preferably 1 hour or more, more preferably 12 hours or more, still more preferably 24 hours or more and particularly preferably 72 hours or more. In addition, the storage time may also be 168 hours or more. For example, by storing the stool sample for at least 12 hours after the mixing, the adverse effects caused against degradation of a nucleic acid by protease generally contained in the stool can be adequately inhibited.

If the stool sample is not stored for a certain amount of time prior to a nucleic acid extraction step from it, in the step of recovering the stool-derived solid fraction, the protease inhibitor is depleted from nucleic acids derived from the stool before the protease inhibitor has sufficiently affected proteases derived from the stool, and thus there is the risk that nucleic acids in the stool-derived solid fraction are easily decomposed by protease activities remaining therein. On the other hand, when the stool sample is stored for a certain amount of time prior to a nucleic acid extraction step from it, after depleting the protease inhibitor in the step of recovering the stool-derived solid fraction, the degradation of nucleic acids in the stool-derived solid fraction is adequately inhibited because activities of proteases derived from the stool are completely lost.

The condition of the storage of the stool sample which obtained from mixing the stool and the solution for preparing the stool sample is not particularly limited as long as it is a condition that allows the obtaining of the improved effect of nucleic acid preservation due to the protease inhibitor in the solution for preparing a stool sample. In this invention, the mixture of the stool and the solution for preparing a stool sample is preferably stored in an environment at a relatively high temperature such as at room temperature rather than in a refrigerator. More specifically, the storage temperature of the stool sample (the mixture) is preferably 10° C. or higher and more preferably 20° C. or higher. The inhibitory effect of the protease inhibitor on protease derived from the stool is obtained to a greater degree when the temperature at which the stool sample is stored is high than when it is low. The reason for this is speculated that the higher the temperature of the storage of the stool sample, the more quickly the water-soluble organic solvent penetrates into the stool. However, the storage temperature is also preferably 50° C. or lower. The reason for this is that there is the risk of the concentration of the water-soluble organic solvent in the stool sample decreasing below the concentration sufficient for demonstrating an improved effect of nucleic acid preservation due to volatilization and the like as a result of storing for a long period of time under a temperature of 50° C. or higher.

As mentioned above, in the method for preparing a stool sample according to the present invention, the better improved effect of nucleic acid preservation can be demonstrated provided the storage temperature of the stool sample is within a range of 10 to 50° C. The storage of the stool sample may be carried out in an environment for which the temperature is controlled using a thermostat and the like, it may also be carried out at room temperature without requiring a special, temperature-controlled environment. Thus, even in the case in which, for example, a stool sample prepared by the method for preparing a stool sample according to the present invention is transported in the absence of temperature control, or in the case in which the stool sample is stored at a relatively high temperature such as at room temperature, an improved effect of nucleic acid preservation can be adequately obtained.

Thus, the method for preparing a stool sample according to the present invention is an extremely preferable sample for preparation of a stool sample in routine health examinations and the like. As mentioned above, nucleic acids in stool are easy to be decomposed. In cases in which the location where a person collecting a stool prepares a stool sample and the location where nucleic acid extraction from it is carried out are separated by a certain distance such as routine health examinations, it is difficult to obtain reliable analysis results from the stool sample because the degradation of nucleic acids and the like proceeds. In order to prevent nucleic acids from being decomposed, a stool sample is stored and transported in low-temperature environments such as in a refrigerator and in a freezer, but it needs special equipment such as a thermostat and high costs of transportation and the like. On the other hand, in the stool sample prepared by the method for preparing a stool sample according to the present invention, the transport time thereof can be considered to be the storage time for enhancing an improved effect of nucleic acid preservation regardless of whether or not the temperature is controlled provided the temperature during transport is within a range of 10 to 50° C.

The stool sample prepared by the method for preparing a stool sample according to the present invention, that is, the stool sample of the present invention, improves effectively the preservation efficiency of nucleic acids contained in stool, especially nucleic acids derived from mammalian cells and the like which are present relatively a little in stool due to protease inhibitory actions achieved by the protease inhibitor, or due to the dehydrating actions, protein denaturing actions and nucleic acid decomposition inhibitory actions achieved by the water-soluble organic solvent. Thus, when a stool sample is prepared by the method for preparing a stool sample according to the present invention, highly reliable analysis results can be expected to be obtained by using the stool sample having been stored for a long period or having been transported, as well as by using the stool sample immediately after preparation. In particular, nucleic acids present in stool, and particularly nucleic acids derived from mammalian cells, can be stably preserved at room temperature for an extended period of time while minimizing changes over time in molecular profiling of mammalian cells such as the cells exfoliated from the large intestine contained in the stool. Consequently, by preparing a collected stool using the method for preparing a stool sample according to the present invention, even in cases in which time is required from stool collection to nucleic acid analysis or in cases in which the location where the stool sample is collected is a considerable distance away from the location where nucleic acids are analyzed, such as screening examinations including routine health examinations, the stool sample can be stored or transported while inhibiting decomposition of nucleic acids, and particularly decomposition of fragile RNA. In addition, special equipment for refrigerating or freezing and the setting of storage temperature conditions are not required, and stool samples can be stored or transported easily and at low cost.

The stool sample of the present invention can be applied to various nucleic acid analyses in the same manner as other biological samples containing nucleic acids. It is particularly preferably used in nucleic acid analyses for investigating the development of cancer or infectious diseases for which early detection is important. In addition, it is preferably used in nucleic acid analyses for investigating for the development of inflammatory diseases such as colitis, enteritis, gastritis or pancreatitis. It may also be used for testing for protruding lesions such as polyps as well as testing for diseases of the large intestine, small intestine, stomach, liver, gallbladder and bile duct, such as gastric ulcer.

In particular, when analyzing, as the target nucleic acid, nucleic acids derived from an organism other than indigenous intestinal bacterium, in other words, the nucleic acid contained in a stool in a relatively small amount as compared to the nucleic acid derived from indigenous intestinal bacterium which are contained therein in a large amount, it is preferable to prepare a stool sample using the solution for preparing of the present invention. Nucleic acids in stool are gradually lost over time following the stool excretion due to degradation or the like. For this reason, when the target nucleic acids are those that are present in stool in a small amount, if an analysis is performed using a stool sample in which the degradation of nucleic acids has already taken place, it may not be possible to recover a sufficient amount of target nucleic acids for the analysis. Accordingly, it is highly probable that the results would appear negative (i.e., the target nucleic acids are absent in the stool), even if the target nucleic acids were present in the stool immediately after the stool excretion. By preparing a stool sample using the solution for preparing of the present invention, the nucleic acids in the stool can be stably preserved, as a result of which the nucleic acids in the stool can be sufficiently recovered even if they are present therein in a small amount, thereby improving the reliability of nucleic acid analysis.

Examples of the above-mentioned nucleic acids derived from an organism other than indigenous intestinal bacterium include nucleic acids derived from mammalian cells, such as nucleic acids derived from cancer cells, and nucleic acids from causative microorganisms responsible for infectious diseases in the early stage or late stage of those infectious diseases, such as hepatitis viruses. In addition, the nucleic acids may be derived from parasites.

Note that in the present invention, the term “indigenous intestinal bacterium/bacteria” refers to the bacterial cells which are relatively abundant in stool and are usually living inside the intestines of animals such as humans. Examples of such indigenous intestinal bacteria include obligate anaerobes such as those belonging to the genera of Bacteroides, Eubacterium, Bifidobacterium and Clostridium; and facultative anaerobes such as those belonging to the genera of Escherichia, Enterobacter, Klebsiella, Citrobacter and Enterococcus

It is possible to examine the development of cancers, such as colon cancer and pancreatic cancer, for example, by detecting and analyzing the nucleic acids derived from cancer cells, in other words, the nucleic acids that are carrying mutations and the like, from the stool sample. In addition, by examining whether the nucleic acids derived from causative microorganisms responsible for the infectious diseases, such as the nucleic acids derived from viruses or the nucleic acids derived from parasites, can be detected or not from the stool sample, it is possible to examine the development of infectious diseases or the presence and absence of parasites. In particular, by using the stool sample for the detection of causative microorganisms excreted in the stool, such as hepatitis A and E viruses, a test for infectious diseases can be carried out in a noninvasive, simple and easy manner. In addition, by examining whether the nucleic acids derived from pathogenic bacteria other than indigenous intestinal bacteria, for example, bacteria causing food poisoning such as enterohemorrhagic Escherichia coli O-157 strain, can be detected or not, development of microbisms can also be tested.

It is particularly preferable to detect a marker indicating neoplastic transformation or a marker indicating an inflammatory gastrointestinal disease. Examples of the marker indicating neoplastic transformation include conventionally known cancer markers, such as carcinoembryonic antigen (CEA) and sialyl Tn antigen (STN), and the presence and absence of mutations in the APC gene, p53 gene, K-ras gene, or the like. Further, detection of methylation of genes, such as p16, hMLHI, MGMT, p14, APC, E-cadherin, ESR1 and SFRP2, is also useful as a diagnostic marker for colon diseases (for example, refer to Lind et al., “A CpG island hypermethylation profile of primary colorectal carcinomas and colon cancer cell lines” Molecular Cancer, 2004, Vol. 3, No. 28). In addition, it has already been reported that the DNA derived from Helicobacter pylori in a stool sample may be used as a marker for gastric cancer (for example, refer to Nilsson et al., Journal of Clinical Microbiology, 2004, Vol. 42, No. 8, pp. 3781-3788). Meanwhile, the Cox-2 gene or the like, for example, is known as a marker indicating inflammatory gastrointestinal disease. Cox gene is also used as a marker indicating neoplastic transformation.

Various kinds of materials are present in the stool sample, and a large number of substances which may become inhibiting factors in the nucleic acid analyses are also present therein. For this reason, it is possible to further improve the analytical accuracy by first recovering the nucleic acids from the stool sample and then performing the nucleic acid analyses using the recovered nucleic acids. As mentioned above, since nucleic acids can be recovered highly efficiently from the stool sample prepared by the method for preparing a stool sample of the present invention, the sample is highly suitable, not only for the analysis of nucleic acids derived from indigenous intestinal bacteria which are present in the stool in large numbers, but also for the analysis of nucleic acids derived from mammalian cells which are present in a small amount. Since the sample is formed of stool, it is preferably used for the analysis of nucleic acids derived from cells of gastrointestinal tracts, such as the large intestine, small intestine and stomach, and it is particularly preferable that the nucleic acids derived from cells exfoliated from the large intestine be analyzed using the sample.

The method for recovering nucleic acids from stool samples is not particularly limited, and any type of method may be adopted as long as it is a method generally used when recovering nucleic acids from samples. The stool sample of the present invention contains mainly nucleic acids derived from an organism other than indigenous intestinal bacterium, such as mammalian cells (hereafter, may be referred to as “mammalian cells or the like”), and nucleic acids derived from indigenous intestinal bacterium. In the nucleic acid recovery from stool samples, although nucleic acids derived from mammalian cells or the like and nucleic acids derived from indigenous intestinal bacteria may be recovered separately, it is particularly preferable to recover them simultaneously. Simultaneously recovering nucleic acids derived from mammalian cells or the like and nucleic acids derived from indigenous intestinal bacteria allows nucleic acids derived from indigenous intestinal bacteria which are highly abundant in stool to function as carriers. As a result, nucleic acids derived from mammalian cells or the like which are present in small numbers can be recovered much more efficiently, as compared to the cases where the nucleic acids are recovered following the isolation of mammalian cells or the like from the stool. Note that nucleic acids recovered from stool samples may be DNA, RNA, or a mixture of DNA and RNA.

For example, nucleic acids derived from mammalian cells or the like and nucleic acids derived from indigenous intestinal bacteria can be recovered simultaneously from the stool sample of the present invention by performing, as a step (a), denaturing of a protein in the stool sample of the present invention, thereby extracting nucleic acids from mammalian cells or the like and indigenous intestinal bacteria in the stool sample; and then, as a step (b), recovery of the extracted nucleic acids.

The denaturing of proteins in the stool sample in the step (a) can be carried out using a conventionally known technique. For example, by adding a compound generally used as a denaturing agent of proteins, such as a chaotropic salt, an organic solvent or a surface active agent, to the stool sample, proteins in the stool sample can be denatured. As the chaotropic salt or surface active agent to be added to the stool sample in the step (a), the same chaotropic salts and surface active agents as those mentioned earlier to be added to the solution for preparing of the present invention can be used. Phenol is preferable as the above organic solvent. Phenol may be neutral or acidic. When acidic phenol is used, it is possible to selectively extract RNA rather than DNA in an aqueous layer. When adding a chaotropic salt, an organic solvent, a surface active agent or the like to the stool sample in the step (a), one type of compound may be added, or two or more types of compounds may be added.

Following the step (a) and prior to the step (b), as a step (c), the protein denatured in the step (a) may be removed. By removing the denatured proteins before recovering nucleic acids, it is possible to improve the quality of recovered nucleic acids. The removal of proteins in the step (c) can be carried out using a conventionally known technique. For example, denatured proteins can be removed by precipitating the denatured proteins by centrifugation, followed by the collection of supernatant alone. Rather than simply performing a centrifugal separation process, denatured proteins can even more thoroughly removed by first adding chloroform to a sample, and subsequently stirring and mixing the resultant sufficiently using a vortex mixer or the like, and the denatured proteins are then precipitated by centrifugation, followed by the collection of supernatant alone.

The recovery of the extracted nucleic acids in the step (b) can be carried out by a known technique such as an ethanol precipitation method and a cesium chloride ultracentrifugation method. Moreover, nucleic acids can be recovered by first, as a step (b1), making the nucleic acids extracted in the step (a) to adsorb to an inorganic substrate; and then, as a step (b2), eluting the nucleic acids adsorbed in the step (b1) from the inorganic substrate.

As the inorganic substrate to which nucleic acids are adsorbed in the step (b1), a conventionally known inorganic substrate which is capable of adsorbing nucleic acids can be used. In addition, the shape of the inorganic substrate is not particularly limited, and it may be a particulate form or a membranous form. Examples of the inorganic substrate include silica-containing particles (beads) such as silica gel, siliceous oxide, glass and diatomaceous earth; and porous membranes made of nylon, polycarbonate, polyacrylate, and nitrocellulose.

As a solvent for eluting the adsorbed nucleic acids in the step (b2) from the inorganic substrate, a solvent generally used for eluting nucleic acids from conventionally known inorganic substrates can be used, where appropriate, determined in consideration of the type of recovered nucleic acids or the method for the following nucleic acid analysis. Purified water is particularly preferable as the solvent for elution. Furthermore, it is preferable to wash the inorganic substrate to which nucleic acids are adsorbed with an appropriate washing buffer, following the step (b1) and prior to the step (b2).

In the case a stool sample is prepared using a solution for preparing a stool sample which contains a chaotropic salt or a surface active agent at a concentration sufficient for extracting nucleic acids from mammalian cells or the like, the step (a) can be omitted in the recovery of nucleic acids from the stool sample.

When a stool sample is prepared using a solution for preparing a stool sample which does not contain a chaotropic salt or a surface active agent at a concentration sufficient for eluting nucleic acids from mammalian cells or the like, as a step (d), it is preferable to recover a solid component from the stool sample prior to the step (a). In order to rapidly mix the stool with the solution for preparing a stool sample, the stool sample contains a larger proportion of liquid components with respect to the solid components, which are derived from the stool. Accordingly, by removing the liquid components from the stool sample and then recovering only the solid components containing mammalian cells or the like and indigenous intestinal bacteria, it is possible to reduce the scale of the samples used for recovering and analyzing nucleic acids. Moreover, by removing a water-soluble organic solvent from the solid components, it is also possible to suppress the adverse effects of the water-soluble organic solvent in the step for recovering nucleic acids from the solid components. For example, by centrifuging the stool sample of the present invention to precipitate the solid components therein and then removing the supernatant, the solid components alone can be recovered. Alternatively, it is also possible to recover the solid components alone by a filtration process or the like. Further, it is also preferable to wash the recovered solid components with an adequate buffer such as phosphate buffered saline (PBS, pH 7.4).

Furthermore, although a denaturing agent of proteins, such as a chaotropic salt, may be added directly to the recovered solid components, it is preferable to first suspend the solid components in an adequate medium and then add a denaturing agent of proteins thereto. When recovering DNA, as an extraction agent, for example, a phosphate buffer, a tris buffer, or the like can be used. It is preferable that DNases in the extraction agent be deactivated by high pressure steam sterilization or the like, and it is more preferable that the extraction agent contains a protease such as Proteinase K. On the other hand, when recovering RNA, as the extraction agent, for example, a citrate buffer or the like can be used. However, since RNA is a material which is highly prone to degradation, it is preferable to use a buffer containing an RNase inhibitor, such as guanidine thiocyanate and guanidine hydrochloride.

Depending on the analytical methods used afterwards, the recovery of nucleic acids from the stool sample may not be needed. More specifically, after extracting nucleic acids from mammalian cells or the like and indigenous intestinal bacteria in the stool sample, the sample can be directly used for the nucleic acid analysis. For example, when pathogenic bacteria and the like are present in large numbers in a stool sample and if the nucleic acids from the pathogenic bacteria were to be analyzed, it is possible to detect genes or the like derived from pathogenic bacteria by first recovering a solid components from the stool sample and then adding thereto an extraction agent, such as PBS, which contains a protease, such as Proteinase K, to mix, and finally using the obtained uniform solution of stool sample directly for the nucleic acid analysis. Alternatively, the recovery of nucleic acids from the stool sample can also be carried out by using a commercially available kit such as a nucleic acid extraction kit or a virus detection kit.

The nucleic acids recovered from the stool sample of the present invention can be analyzed using a conventionally known analytical method. Examples of the method for analyzing nucleic acids include a method for quantitating nucleic acids and a method for detecting specific base sequence regions using polymerase chain reaction (PCR) or the like. In addition, when RNA is recovered, it is possible to first synthesize cDNA by reverse transcriptase reaction, and then analyze the synthesized cDNA in the same manner as described above for the DNA analysis. For example, by detecting the presence or absence of a base sequence region containing a cancer gene or the like or a base sequence region containing microsatellites, it is possible to examine the development of cancers. When using the DNA recovered from the stool sample, for example, the analysis of mutations in the DNA or the analysis of epigenetic changes can be performed. Examples of the mutation analysis include the analyses of insertion, deletion, substitution, duplication and inversion of one or more bases. Examples of the analysis of epigenetic changes include the analyses of methylation and demethylation. On the other hand, when using the recovered RNA, for example, it is possible to detect mutations in the RNA, such as the insertion, deletion, substitution, duplication and inversion of one or more bases, and splicing variants (isoforms). In addition, the analyses of functional RNA (non-coding RNA), such as the analyses of, for example, transfer RNA (tRNA), ribosomal RNA (rRNA) and microRNA (miRNA), can be carried out. Furthermore, the level of RNA expression can also be detected and analyzed. It is particularly preferable to perform an mRNA expression analysis, a mutation analysis of K-ras gene, an analysis of DNA methylation, or the like. These analyses can be carried out according to the methods which are conventionally known in this field. Moreover, it is also possible to use a commercially available analysis kits such as a K-ras gene mutation analysis kit and a methylation detection kit.

In this manner, nucleic acids present in stool can be analyzed with high sensitivity and high accuracy by using the method for preparing a stool sample according to the present invention, the method for recovering nucleic acids from a stool sample prepared according to this preparation method, and a nucleic acid analysis method that uses nucleic acids recovered according to this nucleic acid recovery method. Consequently, this can be expected to contribute and be applicable to early detection and diagnosis of various symptoms and diseases, including colon cancer, observation of the course of treatment, and pathological research on other abnormal states and the like.

By collecting stool in a stool collection container in which the solution for preparing of the present invention is contained in advance, a collected stool can be prepared in an even more simple and rapid manner. In addition, by using a kit for collecting stool that includes both the solution for preparing of the present invention and a stool collection container containing the solution for preparing a stool sample, the effects of the present invention can be achieved more easily. Note that the kit for collecting stool may include a constituent other than the solution for preparing a stool sample and the stool collection container containing the solution, such as a stool collection rod, where appropriate.

The form or size of such stool collection container is not particularly limited, and known stool collection containers which may be able to contain a solvent can be used. A stool collection container in which the lid of the stool collection container and a stool collection rod are integrated into a single unit is preferable because it is easy to handle. In addition, because the amount of stool collected can be controlled, the stool collection rod which is able to collect a predetermined fixed amount of stool is more preferable. Examples of such a stool collection container which is already known include a stool collection container disclosed in Japanese Examined Patent Application, Second Publication No. H6-72837.

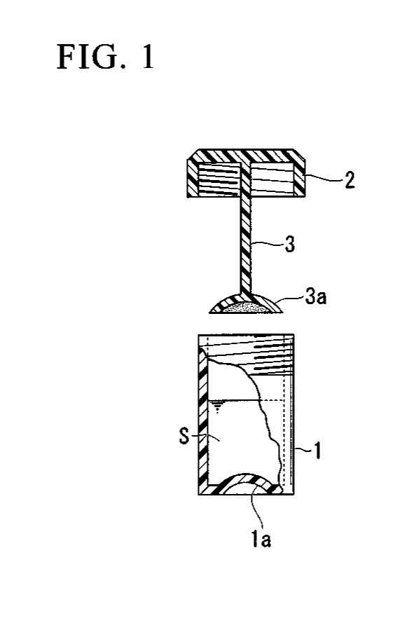

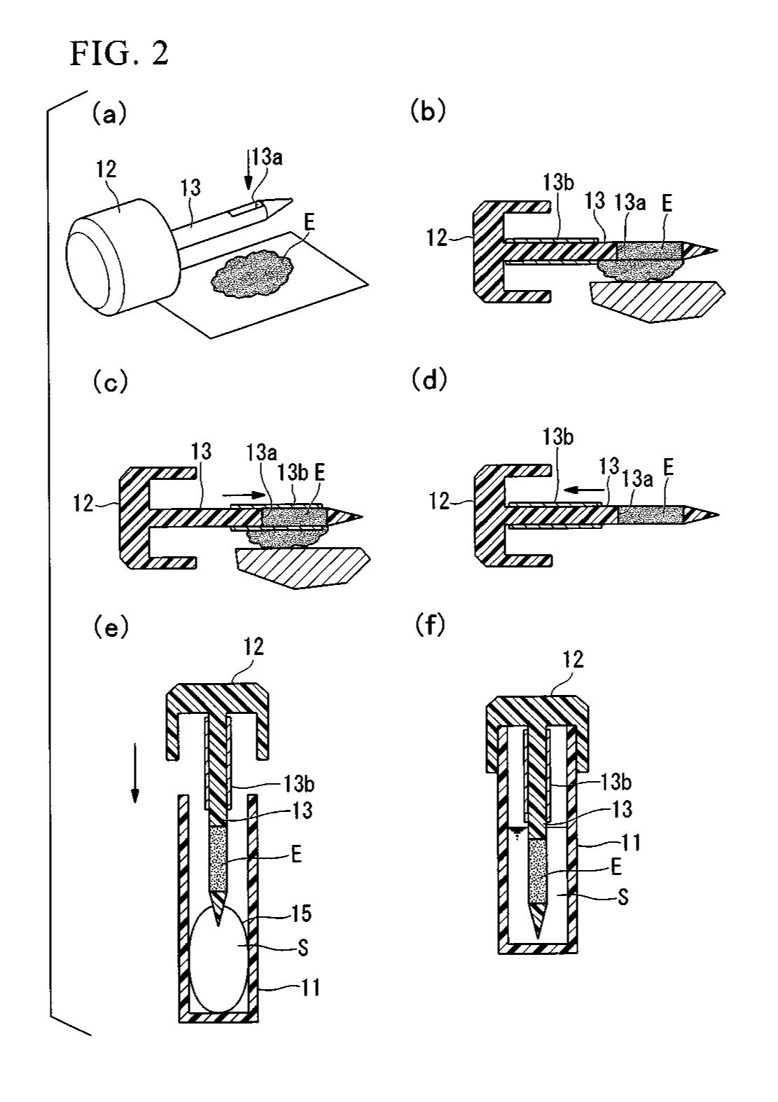

FIGS. 1 and 2 are diagrams showing one aspect of a stool collection container which can be used for a kit for collecting stool according to the present invention. It should be noted that the stool collection containers which can be used for a kit for collecting stool according to the present invention are not limited to these stool collection containers.

First, a stool collection container in FIG. 1 will be described. The stool collection container includes a lid 2 which is integrated with a stool collection rod 3, and a container body 1, and contains the solution S for preparing a stool sample according to the present invention therein. A cup 3a which may collect a predetermined amount of stool is attached to the top end of the stool collection rod 3, and the cup 3a has sieve mesh. Meanwhile, a protruded portion 1a having a shape which is complementary to that of the cup 3a is present in the bottom of the container body 1. By fitting the cup 3a with the protruded portion 1a, the stool collected in the cup 3a is mechanically extruded from the sieve mesh in the cup 3a, and thus the stool can be rapidly dispersed in the solution S for preparing a stool sample.

The stool collection container depicted in FIG. 2 is a stool collection container that includes a lid 12 integrated with a stool collection rod 13 having a pointed end; a container body 11; and a bag 15, which is sealed and contains the solution S for preparing a stool sample according to the present invention, inside the container body 11. An orifice 13a for collecting a certain amount of stool E is formed in the stool collection rod 13. In addition, a movable lid 13b which may become a lid for the orifice 13a by sliding over the stool collection rod 13 is also attached. As shown in FIG. 2a, the movable lid 13b is first slid to the lid 12 side across the orifice 13a so as to leave the orifice 13a in a completely open state, and then the stool collection rod 13 is pressed against the stool E. Then, as shown in FIG. 2b, the orifice 13a is filled with the stool E. In this state, the movable lid 13b is slid to cover the orifice 13a, thereby accurately collecting in an amount equal to the volume of the slot 13a (FIG. 2c). Thereafter, the movable lid 13b is returned to the original position so as to make the orifice 13a in a completely open state (FIG. 2d), and then the lid 12 is housed in the container body 11 (FIG. 2e). When the stool collection rod 13 is housed in the container body 11, because the pointed end of the stool collection rod 13 breaks the bag 15 containing the solution S for preparing a stool sample, the solution S for preparing a stool sample and the stool E are mixed. Since such a stool collection container is filled with a solution only after the stool collection rod is placed inside the container, even when using a solution for preparing a stool sample which is harmful for the human body, such as methanol, accidents due to the solution leakage can be avoided, and thus the container can be handled safely even in the home.

As mentioned above, the solution for preparing of the present invention has superior preservation of nucleic acids contained in stool. Moreover, the solution is able to improve the preservation of cells and cell-derived components (biological components which are present in cells), such as and proteins, as well as the preservation of nucleic acids. For this reason, a stool sample prepared using the solution for preparing of the present invention can be used, not only for the analysis of nucleic acids, but also for the morphological analysis of cells contained in stool, for the analysis of proteins contained in stool, and the like.

Preferred embodiments of the present invention are explained above, but the present invention is not limited to these embodiments. Additions, omissions, replacement, and other modifications in the constitution can be made without departing from the spirit or scope of the present invention. Other than this, the invention is not restricted by the above description, but only by the scope of the appended claims.

Next, the present invention will be described in more detail based on a series of examples, although the scope of the present invention is in no way limited by the following examples. Note that “%” refers to “% by volume (vol %)”, unless otherwise specified. In addition, Caco-2 cells, which were cultured cells, were cultured by ordinary methods.

### EXAMPLE 1

Stool collected from one healthy individual was dispensed into five 15-mL polypropylene tubes (0.5 g each). To each stool, 10 mL of distillated water (Stool Sample 1-1), a 100-times dilution (a solution prepared by diluting the liquid concentrate by 100 times with distillated water) of the protease inhibitor cocktail (manufactured by Sigma-Aldrich Corporation) (Stool Sample 1-2), a 20 mM DTT solution (Stool Sample 1-3), a 5 M Urea solution (Stool Sample 1-4), or a 0.5 M EDTA solution (Stool Sample 1-5) was added respectively, as the solution for preparing a stool sample, and dispersed well to prepare Stool Sample 1-1 to 1-5.

After storing these stool samples for 7 days at 25° C., RNA was recovered from each stool sample. The recovery of RNA from the stool samples was specifically conducted as follows. The solid components of the stool were recovered by centrifuging each tube. a phenol mixture “Trizol” (manufactured by Invitrogen Corporation) was added to the obtained solid components, and the samples were sufficiently mixed using a homogenizer, followed by the addition of chloroform. After sufficiently mixing the resultant by using a vortex mixer, the mixtures were centrifuged (12,000×g) at 4° C. for 20 minutes. The supernatant (aqueous layer) obtained as a result of the centrifugation was passed through an RNA recovery column of the RNeasy Midi Kit (manufactured by Qiagen GmbH). RNA was recovered by carrying out a washing procedure and RNA elution procedure on the RNA recovery column of this kit in accordance with the protocol provided.

RT-PCR was carried out on 1 μg of the recovered RNA to detect the human GAPDH (Glyceraldehyde-3-phosphate dehydrogenase) gene from the recovered DNA. PCR was carried out using the resulting cDNA as a template followed by detection of human GAPDH gene. The GAPDH primer probe MIX (Catalog No.: Hs02786624_gl) was used as primer.

More specifically, 1 μL of the recovered DNA was first dispensed into each well of 96-well PCR plate. Subsequently, 8 μL of ultra-pure water and 10 μL of the nucleic acid amplification reagent “TaqMan Gene Expression Master Mix” (manufactured by Applied Biosystems, Inc.) were added to each well, 1 μL of GAPDH Primer Probe MIX (manufactured by Applied Biosystems, Inc.) were each added thereto and mixed, thereby preparing PCR reaction solutions.

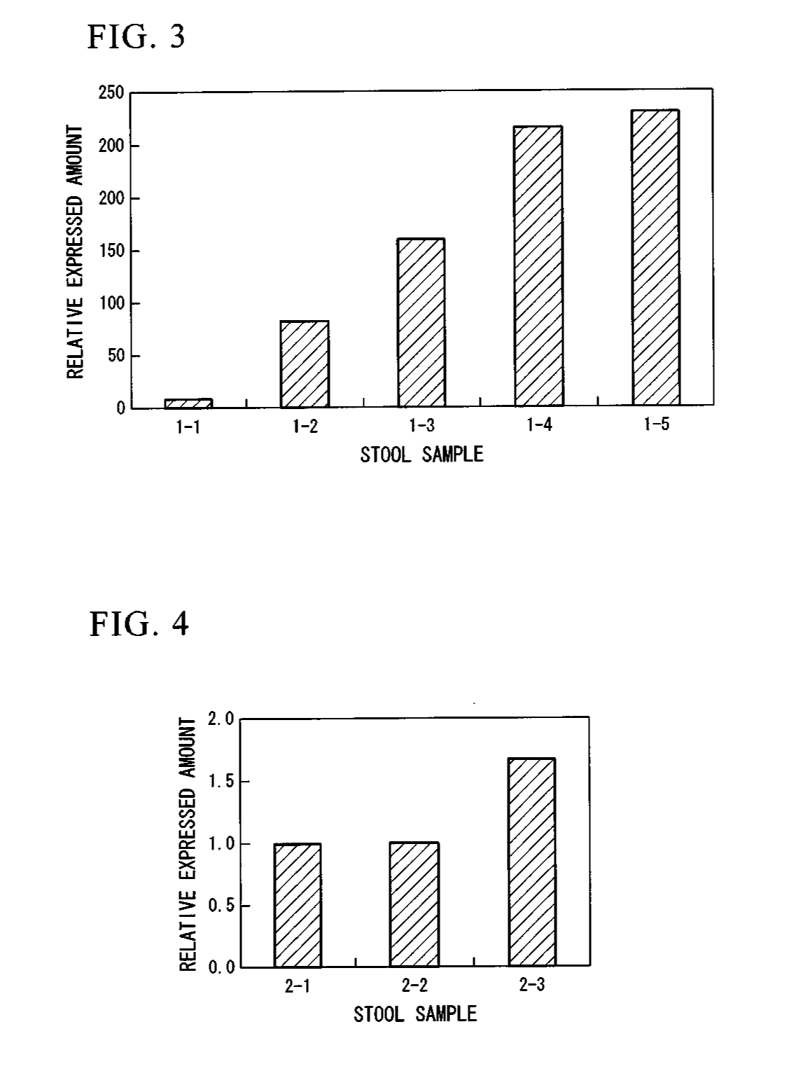

PCR was carried out while measuring fluorescence intensity over time by placing this PCR plate in an ABI real-time PCR apparatus, and initially treating for 10 minutes at 95° C. followed by carrying out 40 cycles of heat cycling consisting of 1 minute at 95° C., 1 minute at 56.5° C. and 1 minute at 72° C., and then further treating for 7 minutes at 72° C. By analyzing the results of fluorescence intensity measurements, the relative values of the expressed amount of GAPDH gene in the RNA recovered from each sample were calculated. The results of a relative comparison of the expressed amounts of GAPDH gene in RNA derived from each stool sample are shown in FIG. 3. The expressed amounts of a GAPDH gene of Stool Sample 1-2 to 1-5, which were prepared by using solutions containing protease inhibitors, were extremely high, at least 10-times higher than that of Stool Sample 1-1, which were prepared by using a solution not containing protease inhibitors. From the above results, it is evident that the preservation of nucleic acids may be spectacularly improved by using the solution for preparing a stool sample having a protease inhibitor (that is, the solution for preparing of the present invention).

### EXAMPLE 2

In the same manner as Example 1, with the exception of using a 60% ethanol solution (pH5.5, Stool Sample 2-1), a 100-times dilution of the protease inhibitor cocktail (manufactured by Sigma-Aldrich Corporation) by a 60% ethanol solution (pH5.5, Stool Sample 2-2), or a 1000-times dilution of the protease inhibitor cocktail (manufactured by Sigma-Aldrich Corporation) by a 60% ethanol solution (pH5.5, Stool Sample 2-3) as the solution for preparing a stool sample, stool samples were prepared and RNA was recovered from the prepared stool samples, and subsequently the relative values of the expressed amount of a GAPDH gene in the RNA recovered from each sample were calculated. The final pH values of all the solutions for preparing a stool sample that were used for preparation of Stool Sample 2-1 to 2-3 were adjusted to 5.5 with a 0.1 M citric acid/sodium hydroxide solution.

The results of a relative comparison of the expressed amounts of GAPDH gene in RNA derived from Stool Sample 2-1 to 2-3 are shown in FIG. 4. From the above results, it is evident that the preservation of RNA may be more improved by using the solution for preparing a stool sample having both a protease inhibitor and a water-soluble organic solvent, and that a protein inhibitor may have an optimum concentration.

### EXAMPLE 3

Stool collected from one colorectal cancer patient who was prospectively confirmed the expression of Cox-2 gene, which is a marker indicating a neoplastic transformation and an inflammatory gastrointestinal disease, was dispensed into three 15-mL polypropylene tubes (0.5 g each). As the solution for preparing a stool sample, a 60% ethanol solution (pH5.5, Stool Sample 3-1), a 100-times dilution of the protease inhibitor cocktail (manufactured by Sigma-Aldrich Corporation) by a 60% ethanol solution (pH5.5, Stool Sample 3-2), or a 1000-times dilution of the protease inhibitor cocktail (manufactured by Sigma-Aldrich Corporation) by a 60% ethanol solution (pH5.5, Stool Sample 3-3) was added to each stool respectively, and dispersed well to prepare Stool Sample 3-1 to 3-3. RNA was recovered from the prepared stool samples, and subsequently the relative values of the expressed amount of Cox-2 gene in the RNA recovered from each sample were calculated in the same manner as Example 1. The final pH values of all the solutions for preparing a stool sample that were used for preparation of Stool Sample 3-1 to 3-3 were adjusted to 5.5 with a 0.1 M citric acid/sodium hydroxide solution.

As results of a relative comparison of the expressed amounts of Cox-2 gene in RNA derived from Stool Sample 3-1 to 3-3, it was shown that these expressed amounts were approximately the same relative values as shown in FIG. 4. From these results, it is evident that for detecting a marker indicating a neoplastic transformation or an inflammatory gastrointestinal disease from a stool sample, the preservation of RNA may be more improved by using the solution for preparing a stool sample having both a protease inhibitor and a water-soluble organic solvent, and that a protein inhibitor may have an optimum concentration.

### EXAMPLE 4

Stool collected from one healthy individual was dispensed into nine 15-mL polypropylene tubes (0.5 g each). Immediately after the dispensation, 10 mL of a 100-times dilution (a solution prepared by diluting the liquid concentrate by 100 times with distillated water) of the protease inhibitor cocktail (manufactured by Sigma-Aldrich Corporation) was added to each stool and dispersed well to prepare stool samples. Then, these stool samples were stored statically for 6 hours at −4° C. (Stool Sample 4-1), 0° C. (Stool Sample 4-2), 4° C. (Stool Sample 4-3), 10° C. (Stool Sample 4-4), 20° C. (Stool Sample 4-5), 30° C. (Stool Sample 4-6), 40° C. (Stool Sample 4-7), 50° C. (Stool Sample 4-8), or 60° C. (Stool Sample 4-9), respectively.

After the storage, RNA was recovered from each stool sample at room temperature. More specifically, the solid components of the stool were recovered by centrifuging each tube. Then, a phenol mixture “Trizol” (manufactured by Invitrogen Corporation) was added to the obtained solid components, and the samples were sufficiently mixed using a homogenizer, followed by the addition of chloroform. After sufficiently mixing the resultant by using a vortex mixer, the mixtures were centrifuged (12,000×g) at 4° C. for 20 minutes. After adding sodium acetate and ethanol to each supernatant (aqueous layer) obtained as a result of the centrifugation and stirring, the supernatants were centrifuged to obtain a precipitate from this centrifugal separation, followed by air-drying the precipitates. These precipitates were dissolved in DEPC-treated water to obtain RNA solutions.

Using the ReverTra Ace qPCR RT Kit (manufactured by TOYOBO Co., Ltd.) which is a reverse transcription reaction kit, cDNA was synthesized from a portion of each of the RNA solutions. 12.5 μL of 2× TaqMan PCR Master Mix (manufactured by Perkin-Elmer Applied Biosystems) was added to the cDNA as template, and a human GAPDH forward primer (SEQ ID NO. 1: 5′-GAAGGTGAAGGTCGGAGTC-3′) and human GAPDH reverse primer (SEQ ID NO. 2: 5′-GAAGATGGTGATGGGATTTC-3′) were each added thereto to respective final concentrations in the reaction solution of 900 nmol, to prepare a PCR solution having a final volume of 25 μL. PCR analysis using SYBR Green was then carried out on this PCR solution using the ABI Prism 7700 Sequence detection System (manufactured by Perkin-Elmer Applied Biosystems). PCR was carried out under the thermal cycle conditions consisting of a denaturation cycle at 95° C. for 10 seconds, followed by 45 cycles at 95° C. for 30 seconds, 55° C. for 30 seconds, and 72° C. for 30 seconds. Quantification was carried out based on the results of fluorescence intensity obtained by using a dilution series of known concentrations of a standard plasmid as template.

TABLE 2

| Stool Sample | 4-1 | 4-2 | 4-3 | 4-4 | 4-5 | 4-6 | 4-7 | 4-8 | 4-9 |
|---|---|---|---|---|---|---|---|---|---|
| Storage | −4° C. | 0° C. | 4° C. | 10° | 20° | 30° | 40° | 50° | 60° |
| Temperature | | | | C. | C. | C. | C. | C. | C. |
| Amount of | 0 | 0 | 0 | 0.5 | 1 | 1.1 | 1.2 | 0.4 | 0 |
| nucleic acid | | | | | | | | | |
| amplification | | | | | | | | | |

The results of analysis are shown in Table 2. In the cases of using RNA derived from Stool Sample 4-1 to 4-3 and 4-9 as templates, each amount of amplified PCR products were lower than that of the detection sensitivity. On the other hand, in the cases of using RNA derived from Stool Sample 4-4 to 4-8 as templates, the presence of amplified PCR products was confirmed.

In other words, it was proved that nucleic acids degradation due to effects of protease contained in stool is able to be effectively inhibited in the case when the storage temperature is 10° C. or above, more effectively inhibited in the case when the storage temperature is 20° C. or above especially. On the other hand, in the case when the storage temperature is above 50° C., the presence of amplified PCR products was not confirmed and it was indicated that nucleic acids degradation was accelerated.

From the above results, it was indicated that in the case when the storage temperature of a stool sample is low, the protease inhibitor cocktail (a mixture of peptidic protease inhibitors) in the stool sample is not able to sufficiently act on protease derived from stool and the nucleic acids degradation in subsequent procedure, such as the step of the recovery of the solid components derived from stool and that of RNA extraction, is accelerated by the activity of protease derived from stool.

### EXAMPLE 5

Stool collected from one healthy individual was dispensed into nine 15-mL polypropylene tubes (0.5g each). Immediately after the dispensation, 10 mL of a 100-times dilution (a solution prepared by diluting the liquid concentrate by 100 times with distillated water) of the protease inhibitor cocktail (manufactured by Sigma-Aldrich Corporation) was added to each stool and dispersed well to prepare stool samples, followed by storing these stool samples statically at 20° C. The storage time of each stool sample was 1 minute (Stool Sample 5-1), 10 minutes (Stool Sample 5-2), 1 hour (Stool Sample 5-3), 12 hours (Stool Sample 5-4), 24 hours (Stool Sample 5-5), 36 hours (Stool Sample 5-6), 48 hours (Stool Sample 5-7), 72 hours (Stool Sample 5-8), or 168 hours (Stool Sample 5-9).

After the elapse of each storage time, the RNA recovery from each stool sample and the PCR analysis using SYBR Green were carried out in the same manner as in Example 3. The relative values of the amount of amplified PCR product of these stool samples were calculated, where the relative value of the fluorescence intensity of the PCR solution from Stool Sample 5-5 whose storage time was 24 hours was equivalent 1.

TABLE 3

| Stool | | | | | | | | | |
|---|---|---|---|---|---|---|---|---|---|
| Sample | 5-1 | 5-2 | 5-3 | 5-4 | 5-5 | 5-6 | 5-7 | 5-8 | 5-9 |
| Storage | 1 | 10 | 1 | 12 | 24 | 36 | 48 | 72 | 168 |
| Time | min | min | hr | hr | hr | hr | hr | hr | hr |
| Amount | 0 | 0 | 0 | 0.8 | 1.0 | 1.5 | 1.8 | 1.3 | 1.0 |
| of nucleic | | | | | | | | | |
| acid | | | | | | | | | |
| amplifica- | | | | | | | | | |
| tion | | | | | | | | | |

The results of analysis are shown in Table 3. In the cases of using RNA derived from Stool Sample 5-1 to 5-3 as templates, each amount of amplified PCR products were lower than that of the detection sensitivity. On the other hand, in the cases of using RNA derived from Stool Sample 5-4 to 5-9 as templates, the presence of amplified PCR products was confirmed. In particular, when the storage time were within a rage 12 to 28 hours, as the storage time is longer, the amount of amplified PCR products becomes larger and a certain amount of amplified PCR products was detected even when the storage time is 48 hours.

In other words, when the storage time is 12 hours or over, it was proved that nucleic acids degradation due to effects of protease contained in stool is able to be effectively inhibited.

From these results, it was indicated that in the case when the storage of a stool sample is omitted or several minutes, the protease inhibitor cocktail (a mixture of peptidic protease inhibitors) in the stool sample is not able to sufficiently act on protease derived from stool and the nucleic acids degradation in subsequent procedure, such as the step of the recovery of the solid components derived from stool and that of RNA extraction, is accelerated by the activity of protease derived from stool.

### REFERENCE EXAMPLE 1

Stool collected from one healthy individual was dispensed into three 15-mL polypropylene tubes (1.0 g each). Immediately after the dispensation, one polypropylene tube was quickly subjected to a freezing treatment using liquid nitrogen, thereby preparing a stool sample (1A). After the dispensation, 10 mL of 70% ethanol solution was added to one of the other polypropylene tubes. After sufficiently dispersing the stool in the solution, the tube was left statically for 1 hour, thereby preparing a stool sample (1B). After the dispensation, the remaining one polypropylene tube was quickly transferred to an extraction step without adding any solutions or the like thereto, thereby preparing a stool sample (1C).

Thereafter, RNA was recovered from each stool sample. More specifically, 3 mL of a phenol mixture “Trizol” (manufactured by Invitrogen Corporation) was added to each stool sample, and the samples were sufficiently mixed for 30 seconds or more using a homogenizer, followed by the addition of 3 mL of chloroform to each stool sample. After sufficiently mixed by using a vortex mixer, the mixtures were centrifuged (12,000×g) at 4° C. for 20 minutes. The supernatant (aqueous layer) obtained as a result of the centrifugation was passed through an RNA recovery column of the RNeasy midi kit (manufactured by Qiagen GmbH), and RNA was recovered by the washing of the RNA recovery column followed by RNA extraction according to the protocol provided in the kit. The recovered RNA was quantified using the NanoDrop instrument (manufactured by NanoDrop Technologies, Inc.).

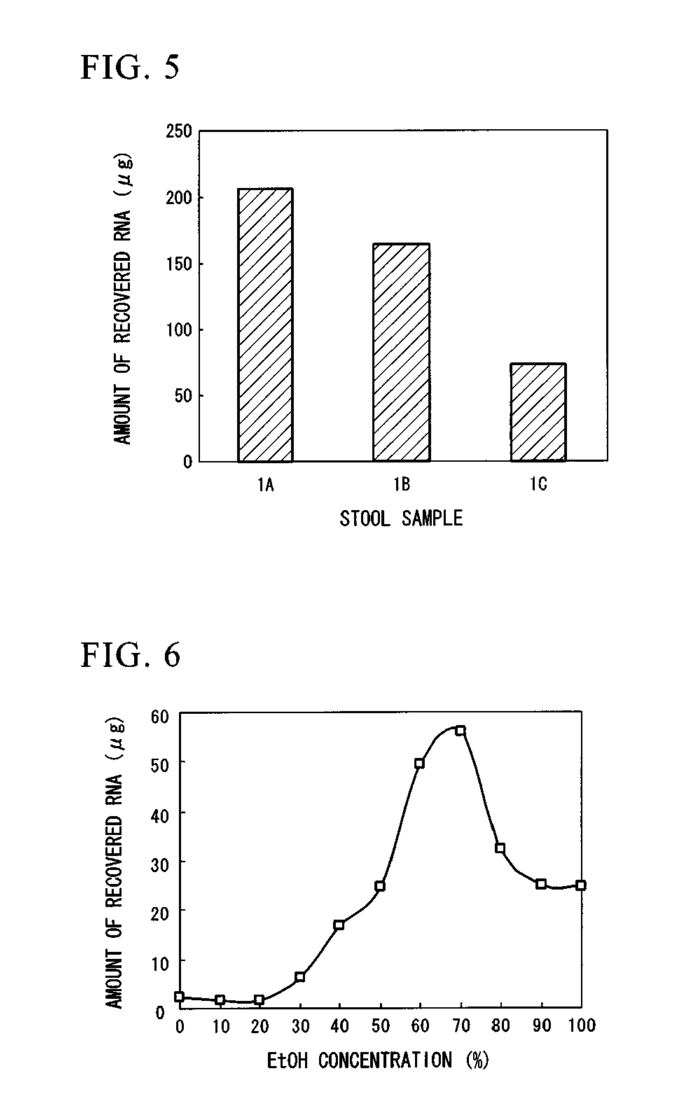

FIG. 5 is a diagram showing the amount of RNA recovered from each stool sample. From the stool sample (1B) prepared using an ethanol solution which was the solution of the present invention for preparing a stool sample, it was possible to recover a much larger amount of RNA, as compared to the stool sample (1C) in which nucleic acids were quickly extracted immediately after the stool collection, although it was slightly less than the amount of RNA recovered from the stool sample (1A) which was subjected to a freezing treatment immediately after the stool collection. From these results, it is evident that even when a preparation process is conducted at room temperature, by using the solution for preparing a stool sample according to the present invention in the preparation process, it is possible to obtain a stool sample from which nucleic acids may be recovered highly efficiently. In those cases where a patient is collecting stool at home for a checkup or the like, it is desirable that the preparation of stool samples can be carried out at a temperature close to room temperature. The solution for preparing a stool sample according to the present invention fully satisfies such a requirement.

### REFERENCE EXAMPLE 2

0.5 g of stool from one healthy individual was mixed with 5.0×10⁵ cells of a human colon cancer cell line (Caco-2 cells) which were expressing a high level of MDR1 (multidrug resistance 1) gene to prepare an artificial stool of colon cancer patients, and this artificial stool was used to prepare stool samples by the method for preparing a stool sample according to the present invention.

More specifically, the artificial stool of colon cancer patients was dispensed into 15-mL polypropylene tubes (0.5 g each), and the solutions for preparing a stool sample indicated in Table 4 were added to each tube and mixed, thereby preparing the stool samples. Note that the “universal collection medium” in the table refers to a preservation medium disclosed in Japanese Translation of PCT Application No. 2004-500897 which contains 500 mL of Puck's Saline G, 400 mg of sodium bicarbonate, 10 g of bovine serum albumin (B SA), 500 units/L of penicillin G, 500 mg/L of streptomycin sulfate, 1.25 mg/L of amphotericin B and 50 mg/L of gentamicin. The prepared stool samples were preserved in a constant temperature incubator set at room temperature (25° C.) for 1, 3, 7, and 10 days, respectively.

TABLE 4

| Solution for preparing stool sample | |
|---|---|
| (2A) | 5 mL of 70% methanol solution |
| (2B) | 1 mL of 100% methanol solution |
| (2C) | 5 mL of universal collection medium |
| (2D) | 5 mL of PBS |

Following preservation, RNA was recovered from each stool sample, and attempts were made in order to detect the transcription products (mRNA) of MDR1 gene from the recovered RNA. With respect to the stool sample prepared using the solution for preparing a stool sample (2C) (hereafter, referred to as the “stool sample (2C)”), mammalian cells including Caco-2 cells were first separated, followed by the RNA recovery. With respect to the stool samples prepared using the solutions for preparing a stool sample other than the solution for preparing a stool sample (2C), the nucleic acids originating from mammalian cells and the nucleic acids originating from bacteria were recovered simultaneously without the separation of mammalian cells. The separation of mammalian cells from the stool sample (2C) was specifically conducted as follows. 5 mL of Histopack 1077 solution (manufactured by Sigma-Aldrich Corporation) was added to the stool sample (2C) and mixed, and the mixture was then centrifuged (200×g) at room temperature for 30 minutes, followed by the recovery of the interfacial portion between the suspension and the Histopack 1077 solution. The separated mammalian cells were washed three times with PBS.

The recovery of RNA from the stool samples was specifically conducted as follows. 3 mL of a phenol mixture “Trizol” (manufactured by Invitrogen Corporation) was first added to the stool sample (or to the separated mammalian cells, only for the case of the stool sample (2C)), and the samples were sufficiently mixed for 30 seconds or more using a homogenizer, followed by the addition of 3 mL of chloroform. Then, the resultant was centrifuged at 12,000×g for 10 minutes. The supernatant (aqueous layer) obtained as a result of the centrifugation was collected in a new polypropylene tube. Thereafter, RNA was recovered from the collected supernatant using the RNeasy midi kit (manufactured by Qiagen GmbH).

Reverse transcriptase polymerase chain reaction (RT-PCR) was performed using the recovered RNA, and PCR was then carried out using the obtained cDNA as a template. As primers, a base sequence for amplifying MDR1 gene which had a sequence number 1 and a base sequence for amplifying MDR1 gene which had a sequence number 2 were used as a forward primer and a reverse primer, respectively.

More specifically, to a 0.2-mL PCR tube, 12 μL of ultra-pure water and 2 μL of a buffer (10×) were added, and 1 μL of cDNA, the forward primer, the reverse primer, magnesium chloride, dNTP, and DNA polymerase were each added thereto and mixed, thereby preparing a PCR reaction solution.PCR was carried out for 30 cycles, each amplification cycle consisted of incubating the PCR tubes at 95° C. for 30 seconds, 60° C. for 30 seconds, and then at 72° C. for 1 minute. The PCR products obtained as a result of the amplification was electrophoresed using the Agilent DNA 1000 LabChip (registered trade mark) kit (manufactured by Agilent Technologies, Inc.), and the intensity of the obtained band was measured, thereby examining the extent of amplification indicated by the PCR products.

TABLE 5

| Preservation periods | 1 day | 3 days | 7 days | 10 days |
|---|---|---|---|---|
| Stool sample (2A) | ++ | ++ | ++ | + |
| Stool sample (2B) | ++ | ++ | + | + |
| Stool sample (2C) | − | − | − | − |
| Stool sample (2D) | − | − | − | − |

++: Intense level of amplification; +: Intermediate level of amplification; +/−: Weak level of amplification; −: No amplification

Table 5 summarizes the extent of amplification indicated by the PCR products which originated from each stool samples, based on different preservation periods. Note that in the table, “stool sample (2A)” refers to a stool sample prepared using a solution for preparing a stool sample (2A), “stool sample (2B)” refers to a stool sample prepared using a solution for preparing a stool sample (2B), and “stool sample (2D)” refers to a stool sample prepared using a solution for preparing a stool sample (2D), respectively.

As a result, with respect to the stool sample (2D), although the presence of amplified PCR products was confirmed when the sample preserved for 1 day was used, no amplification was observed when using the samples preserved for 3 days or longer. On the other hand, with respect to the stool samples (2A) and (2B) prepared using a solution for preparing a stool sample (2A) or a solution for preparing a stool sample (2B) which were the solutions for preparing stool samples according to the present invention, the presence of amplified PCR products was confirmed even when the samples preserved for 10 days were used. Meanwhile, with respect to the stool sample (2C) prepared using a solution for preparing a stool sample (2C) disclosed in Japanese Translation of PCT Application No. 2004-500897, no amplification of PCR products was observed even when using the sample preserved only for 1 day.

From the above results, it is evident that from the stool samples prepared by the preparation method according to the present invention, it is possible to efficiently recover nucleic acids contained in stool. In addition, by using the stool samples according to the present invention, it is also apparent that the accuracy for RNA analysis may also be improved. It is thought that this is because by using the solution for stool sample according to the present invention, the nucleic acids originating from mammalian cells that are contained in the stool and even RNA which is particularly prone to degradation, can be stably preserved for a long time at room temperature.

On the other hand, because no amplification of PCR products originating from the stool sample (2C) was observed, when a solution containing an antibiotic was used as the solution for preparing a stool sample, although bacterial cells will be killed by the antibiotic, it is possible that the RNA degradation may even be accelerated due to the release of RNase or the like from the dead bacterial cells. In addition, because the number of mammalian cells contained in stool is small, when the mammalian cells are separated from the stool, as compared to the method for recovering nucleic acids according to the present invention in which the nucleic acids originating from bacterial cells may function as a carrier, it is possible that sufficient amount of nucleic acids may be difficult to recover.

### REFERENCE EXAMPLE 3

Ethanol solutions of 0%, 10%, 20%, 30%, 40%, 50%, 60%, 70%, 80%, 90% and 100% were prepared by dilution using ultra-pure water. 5 mL of each of these ethanol solutions was dispensed into 15-mL polypropylene tube.

After dispensing 0.5 g of stool collected from a healthy individual to each of these tubes, the tubes were left statically at 37° C. for 48 hours. Thereafter, each tube was centrifuged, and the resulting supernatant was removed to obtain a solid component. 3 mL of a phenol mixture “Trizol” (manufactured by Invitrogen Corporation) was added to the obtained solid components, and the samples were sufficiently mixed for 30 seconds or more using a homogenizer, followed by the addition of 3 mL of chloroform. Then, the resultant was centrifuged at 12,000×g for 10 minutes. The supernatant (aqueous layer) obtained as a result of the centrifugation was collected in a new polypropylene tube. Thereafter, RNA was recovered from the collected supernatant using the RNeasy midi kit (manufactured by Qiagen GmbH).

FIG. 6 is a diagram showing the amount of RNA recovered from stool samples prepared using ethanol solutions of each concentration. As a result, it is clear that when an alcohol such as ethanol is used as an active ingredient of the solution for preparing a stool sample, the alcohol concentration is preferably at least 30%, more preferably at least 50%, still more preferably within a range from 50 to 80%, and most preferably within a range from 60 to 70%.

### REFERENCE EXAMPLE 4

Stool collected from five healthy individuals was mixed adequately and was then dispensed into two 15-mL polypropylene tubes (0.2 g each). 1 mL of a 32% modified ethanol solution containing 18% of isopropanol (having a total alcohol concentration of 50%) was added to one of the polypropylene tubes and mixed adequately, and the tube was then left statically at 25° C. for 1 day. The prepared stool sample was used as a stool sample (4A). One of the remaining polypropylene tubes was used as a control sample, and was quickly transferred to a deep freezer set at −80° C. after the dispensation.

DNA was recovered from both stool samples using the QIAamp DNA Stool Mini Kit (manufactured by Qiagen GmbH) which was a DNA extraction kit from stool. The concentration of the recovered DNA was quantified by spectrophotometry. As a result, it was possible to recover almost the same amount of DNA from both stool samples.

A mutation analysis was conducted, using 100 ng of the recovered DNA as well as the “K-ras codon 12 mutations detection reagent” (manufactured by Wakunaga Pharmaceutical Co., Ltd.) which was a kit for analyzing mutations in the K-ras gene, and following the protocol attached to the kit. As a result, the analyses of DNA recovered from the stool sample (4A) against 6 types of mutated genes were all negative, as was the case where the DNA recovered from the control sample was used.

From the above results, it is evident that by using the nucleic acids recovered by the method for preparing a stool sample according to the present invention and the method for recovering nucleic acids according to the present invention, even the analyses of nucleic acids which require a high level of accuracy, such as the analyses of gene mutations, can be carried out with an adequate level of accuracy. In addition, although modified ethanol prepared by mixing isopropanol and ethanol was used in the present example as a process solution, equivalent results were obtained even when a 50% ethanol solution which had the same alcohol concentration as that of the modified ethanol was used.

### REFERENCE EXAMPLE 5

Stool collected from one healthy individual was dispensed into three 15-mL polypropylene tubes (0.1 g each). 3 mL of a 70% ethanol solution was added to one of the polypropylene tubes to sufficiently disperse the stool, and the obtained stool sample was used as a stool sample (10A). On the other hand, to the remaining two polypropylene tubes, 2.4 mL of “ISOGEN” (manufactured by Nippon Gene Co., Ltd.) was each added to sufficiently disperse the stool, and the obtained stool samples were used as comparative samples (P1) and (P2). It should be noted that “ISOGEN” is a phenol-containing material that contains 40% of phenol (having a water solubility of about 10% by weight).

RNA was rapidly recovered from the comparative sample (P1) following the stool dispersion. More specifically, the stool sample was sufficiently mixed for 30 seconds or more using a homogenizer, followed by the addition of 3 mL of chloroform. Then, the resultant was centrifuged at 12,000×g for 10 minutes. The supernatant (aqueous layer) obtained as a result of the centrifugation was collected in a new polypropylene tube. Thereafter, RNA was recovered from the collected supernatant using the RNeasy midi kit (manufactured by Qiagen GmbH).

As for the comparative sample (P2), after statically leaving the sample at room temperature for 5 hours, RNA was recovered from it in the same manner as that described for the comparative sample (P1).

On the other hand, the stool sample (5A) was left statically at room temperature for 5 hours, just like the comparative sample (P2). Then the stool sample (5A) was centrifuged and the resulting supernatant was removed to obtain precipitates (solid components). RNA was recovered in the same manner as that described for the comparative sample (P1), after adding 2.4 mL of “ISOGEN” to the obtained precipitates.

The recovered RNA was quantified using the NanoDrop instrument (manufactured by NanoDrop Technologies, Inc.). As a result, although it was possible to recover 32 μg of RNA from the comparative sample (P1) with which the RNA recovery was conducted immediately after the preparation of stool sample, only 14 μg of RNA was recovered from the comparative sample (P2) with which the RNA recovery operation was conducted after statically leaving the sample at room temperature for 5 hours. On the other hand, from the stool sample (5A), although the RNA recovery operation was conducted after statically leaving the sample at room temperature for 5 hours, it was possible to recover 57 μg of RNA, which was far more than the amount of RNA recovered from the comparative sample (P1).

From these results, it is clear that by using the solution for preparing a stool sample according to the present invention, RNA may be recovered highly efficiently, as compared to the conventional cases where a phenol solution was used.

### INDUSTRIAL APPLICABILITY

According to the stool sample preparation method of the present invention, since a stool sample that allows nucleic acids in the stool sample to be efficiently preserved can be prepared easily, the present invention can be used particularly in fields such as clinical testing, including routine health examinations, using stool samples.

A stool sample in which nucleic acids present in stool can be stored stably can be prepared according to the stool sample preparation method of the present invention. Namely, according to the stool sample preparation method of the present invention, nucleic acids derived from the organism other than indigenous intestinal bacteria contained in relatively small amounts in stool samples, such as nucleic acids derived from mammalian cells, can be maintained in a stable state that enables them to be stored for a long period of time at room temperature. In this manner, use of the stool sample preparation method of the present invention enables collection of stool to preparation, storage and transport of a stool sample to be carried out easily at room temperature while stably storing nucleic acids present in the stool sample, thereby making this extremely preferable for preparation of a stool sample for use in routine health examinations and other screening examinations. Moreover, even in the case of preparing a stool sample for analysis of nucleic acids derived from the organism other than indigenous intestinal bacteria such as mammalian cells, since there is no need for a complex procedure involving separation of the creature or cells thereof and the like, on which detection of mammalian cells and the like is to be carried out, from the stool sample, even in cases of processing a large number of specimens, both labor and costs can be effectively reduced. In particular, a stool sample can be prepared even more easily by using the stool collection kit of the present invention.

### BRIEF DESCRIPTION OF THE REFERENCE SYMBOLS

1 Container body

1a Protrusion

2 Cover

3 Stool collection rod

3a Cup

S Ssolution for preparing a stool sample

11 Container body

12 Cover

13 Stool collection rod

13a Slot

13b Movable cover

15 Pouch

E Stool

### SEQUENCE LISTINGS

PCT International Patent Application No. PCT/JP2009/070186 sequence list

1. A method for preparing a stool sample, comprising: mixing a collected stool with a solution for preparing a stool sample having a protease inhibitor as an active ingredient,
wherein the stool sample is used for analyzing a nucleic acid contained in the stool.

2. The method for preparing a stool sample according to claim 1,
wherein the mixture of the stool and the solution for preparing a stool sample is stored for a predetermined amount of time.

3. The method for preparing a stool sample according to claim 2,
wherein the amount of time during which the mixture is stored is 1 hour or more.

4. The method for preparing a stool sample according to claim 1,
wherein the protease inhibitor is one or more members selected from the group consisting of a peptidic protease inhibitor, a reducing agent, a protein denaturing agent, and chelating agent.

5. The method for preparing a stool sample according to claim 1,
wherein the protease inhibitor is one or more members selected from the group consisting of AEBSF, Aprotinin, Bestain, E-64, Leupeptin, Pepstatin, DTT(dithiothreitol), and EDTA.

6. The method for preparing a stool sample according to claim 1,
wherein the solution for preparing a stool sample further contains a water-soluble organic solvent as an active ingredient.

7. The method for preparing a stool sample according to claim 1,
wherein the solution for preparing a stool sample has a buffering action.

8. The method for preparing a stool sample according to claim 1,
wherein the pH of the solution for preparing a stool sample is from 2 to 6.5.

9. The method for preparing a stool sample according to claim 6,
wherein the water-soluble organic solvent is one or more members selected from the group consisting of a water-soluble alcohol, ketone and aldehyde.

10. The method for preparing a stool sample according to claim 6,
wherein the water-soluble organic solvent is one or more members selected from the group consisting of a water-soluble alcohol and ketone, and the concentration of the water-soluble organic solvent is 30% or more.

11. The method for preparing a stool sample according to claim 6,
wherein the water-soluble organic solvent contains one or more members selected from the group consisting of ethanol, propanol and methanol as water-soluble alcohol.

12. The method for preparing a stool sample according to claim 6,
wherein the water-soluble organic solvent is ethanol.

13. The method for preparing a stool sample according to claim 6,
wherein the water-soluble organic solvent contains one or more members selected from the group consisting of acetone and methyl ethyl ketone as ketone.

14. The method for preparing a stool sample according to claim 6,
wherein the water-soluble organic solvent is an aldehyde, and the concentration of the water-soluble organic solvent is within a range of 0.01 to 30%.

15. The method for preparing a stool sample according to claim 1,
wherein in terms of a mixing ratio of the stool and the solution for preparing a stool sample, a volume of the solution for preparing the stool sample is one or more relative to 1 volume of the stool.

16. The method for preparing a stool sample according to claim 2,
wherein the amount of time during which the mixture is stored is 12 hours or more.

17. The method for preparing a stool sample according to claim 2,
wherein the amount of time during which the mixture is stored is 24 hours or more.

18. The method for preparing a stool sample according to claim 2,
wherein the amount of time during which the mixture is stored is 72 hours or more.

19. The method for preparing a stool sample according to claim 1,
wherein the pH of the solution for preparing a stool sample is from 3 to 6.

20. The method for preparing a stool sample according to claim 1,
wherein the pH of the solution for preparing a stool sample is from 4.5 to 5.5.

21. The method for preparing a stool sample according to claim 1,
wherein the solution for preparing a stool sample further contains a surface active agent.

22. The method for preparing a stool sample according to claim 1,
wherein the solution for preparing a stool sample further contains a colorant.

23. A solution for preparing a stool sample that is used to mix a collected stool, comprising: a protease inhibitor as an active ingredient,
wherein the stool sample being used for recovering a nucleic acid from the stool sample.

24. The solution for preparing a stool sample that is used to mix a collected stool according to claim 23,
wherein the solution for preparing a stool sample further contains a water-soluble organic solvent as an active ingredient.

25. The solution for preparing a stool sample that is used to mix a collected stool according to claim 23,
wherein the protease inhibitor is one or more members selected from the group consisting of a peptidic protease inhibitor, a reducing agent, a protein denaturing agent, and chelating agent.

26. The solution for preparing a stool sample that is used to mix a collected stool according to claim 24,
wherein the water-soluble organic solvent is one or more members selected from the group consisting of a water-soluble alcohol and ketone.

27. A stool collection kit, comprising:
a stool collection container; and
a solution for preparing a stool sample having a protease inhibitor as an active ingredient,
wherein the stool collection container includes the solution for preparing a stool sample.

28. A stool sample prepared by the method for preparing a stool sample according to claim 1.

29. A method for recovering a nucleic acid from a stool sample comprising:
simultaneously recovering a nucleic acid derived from indigenous intestinal bacterium and a nucleic acid derived from an organism other than indigenous intestinal bacterium, from the stool sample, and
the stool sample is prepared by mixing a collected stool with a solution for preparing a stool sample having a protease inhibitor as an active ingredient.

30. The method for recovering a nucleic acid from a stool sample according to claim 29,
wherein the nucleic acid derived from the organism other than indigenous intestinal bacterium is the nucleic acid derived from a mammalian cell.

31. The method for recovering a nucleic acid from a stool sample according to claim 29,
wherein the method comprising:
(a) denaturing a protein in the stool sample and thereby extracting a nucleic acid from indigenous intestinal bacterium and an organism other than indigenous intestinal bacterium in the stool sample; and
(b) recovering the nucleic acid extracted in the step (a).

32. The method for recovering a nucleic acid from a stool sample according to claim 31,
further comprising, following the step (a) and prior to the step (b),
(c) removing the protein denatured in the step (a).

33. The method for recovering a nucleic acid from a stool sample according to claim 31,
wherein denaturing of a protein in the step (a) is carried out using one or more materials selected from the group consisting of a chaotropic salt, an organic solvent and a surface active agent.

34. The method for recovering a nucleic acid from a stool sample according to claim 33,
wherein the organic solvent is phenol.

35. The method for recovering a nucleic acid from a stool sample according to claim 32,
wherein the removal of denatured protein in the step (c) is carried out using chloroform.

36. The method for recovering a nucleic acid from a stool sample according to claim 31,
wherein the recovery of nucleic acid in the step (b) includes:
(b1) adsorbing the nucleic acid extracted in the step (a) to an inorganic support, and
(b2) eluting the nucleic acid adsorbed in the step (b1) from the inorganic support.

37. The method for recovering a nucleic acid from a stool sample according to claim 31,
further comprising, prior to the step (b),
(d) recovering a solid component from the stool sample.

38. A method for analyzing a nucleic acid comprising:
conducting an analysis of a nucleic acid derived from a mammalian cell,
wherein the nucleic acid is recovered from a stool sample by use of the method for recovering a nucleic acid according to claim 29.

39. The method for analyzing a nucleic acid according to claim 38,
wherein the mammalian cell is a gastrointestinal tract cell.

40. The method for analyzing a nucleic acid according to claim 38,
wherein the mammalian cell is a cell exfoliated from a large intestine.

41. The method for analyzing a nucleic acid according to claim 38,
wherein the nucleic acid derived from a mammalian cell is a marker indicating a neoplastic transformation.

42. The method for analyzing a nucleic acid according to claim 38,
wherein the nucleic acid derived from a mammalian cell is a marker indicating an inflammatory gastrointestinal disease.

43. The method for analyzing a nucleic acid according to claim 38,
wherein the nucleic acid derived from a mammalian cell is a nucleic acid derived from COX-2 gene.

44. The method for analyzing a nucleic acid according to claim 38,
wherein the analysis is one or more of RNA analysis and DNA analysis.

45. The method for analyzing a nucleic acid according to claim 44,
wherein the RNA analysis is one or more analysis selected from the group consisting of an analysis for insertion, deletion, substitution, duplication or inversion of one or more bases in the RNA, an analysis for a splicing variant, an mRNA expression analysis, and a functional RNA analysis.

46. The method for analyzing a nucleic acid according to claim 44,
wherein the DNA analysis is one or more of a mutation analysis and an analysis of an epigenetic change.

47. The method for analyzing a nucleic acid according to claim 46,
wherein the mutation analysis is an analysis for one or more mutations of an insertion, deletion, substitution, duplication or inversion of one or more bases.

48. The method for analyzing a nucleic acid according to claim 46,
wherein the analysis of an epigenetic change is one or more of a DNA methylation analysis and a DNA demethylation analysis.

49. The method for analyzing a nucleic acid according to claim 46,
wherein the mutation analysis is a mutation analysis of a K-ras gene.
